SEQUENCE LISTING (31 sequences)

SEQ ID NO: 1 - protein, 13 aa, Artificial Sequence
  Description of Artificial Sequence Synthetic peptide
GWTLNSAGYLLGP

SEQ ID NO: 2 - protein, 30 aa, Homo sapiens
GWTLNSAGYLLGPHAVGNHRSFSDKNGLTS

SEQ ID NO: 3 - protein, 29 aa, Bos taurus
GWTLNSAGYLLGPHALDSHRSFQDKHGLA

SEQ ID NO: 4 - protein, 29 aa, Sus scrofa
GWTLNSAGYLLGPHAIDNHRSFHDKYGLA

SEQ ID NO: 5 - protein, 29 aa, Rattus rattus
GWTLNSAGYLLGPHAIDNHRSFSDKHGLT

SEQ ID NO: 6 - protein, 29 aa, Artificial Sequence
  Description of Artificial Sequence Synthetic peptide
GWTLNSAGYLLGPHAVNHRSFSDKNGLTS

SEQ ID NO: 7 - protein, 123 aa, Homo sapiens
MARGSALLLASLLLAAALSASAGLWSPAKEKRGWTLNSAGYLLGPHAVGNHRSFSDKNGLTSKRELRPEDDMKPGSFDRSIPENNIMRTIIEFLSFLHLKEAGALDRLLDLPAAASSEDIERS

SEQ ID NO: 8 - protein, 123 aa, Bos taurus
MPRGSVLLLASLLLAAALSATLGLGSPVKEKRGWTLNSAGYLLGPHALDSHRSFQDKHGLAGKRELEPEDEARPGSFDRPLAENNVVRTIIEFLTFLHLKDAGALERLPSLPTAESAEDAERS

SEQ ID NO: 9 - protein, 123 aa, Sus scrofa
MPRGCALLLASLLLASALSATLGLGSPVKEKRGWTLNSAGYLLGPHAIDNHRSFHDKYGLAGKRELEPEDEARPGGFDRLQSEDKAIRTIMEFLAFLHLKEAGALGRLPGLPSAASSEDAGQS

SEQ ID NO: 10 - protein, 116 aa, Homo sapiens
MAPPSVPLVLLLVLLLSLAETPASAPAHRGRGGWTLNSAGYLLGPVLHLPQMGDQDGKRETALEILDLWKAIDGLPYSHPPQPSKRNVMETFAKPEIGDLGMLSMKIPKEEDVLKS

SEQ ID NO: 11 - protein, 60 aa, Homo sapiens
APAHRGRGGWTLNSAGYLLGPVLHLPQMGDQDGKRETALEILDLWKAIDGLPYSHPPQPS

SEQ ID NO: 12 - protein, 60 aa, Sus scrofa
APVHRGRGGWTLNSAGYLLGPVLHPPSRAEGGGKGKTALGILDLWKAIDGLPYPQSQLAS

SEQ ID NO: 13 - protein, 60 aa, Rattus rattus
APAHRGRGGWTLNSAGYLLGPVLHLSSKANGGRKTDSALEILDLWKAIDGLRYSRSPRMT

SEQ ID NO: 14 - DNA, 765 bp, Homo sapiens
ccacgcgtccgggacccggcccgcgccttctgcccctgctgccggccgcgccatgcggtgagcgccccaggccgccagagcccacccgacccggcccgacgcccggacctgccgcccagacccgccaccgcacccggaccccgacgctccgaacccgggcgcagccgcagctcaagatggcccgaggcagcgccctccttctcgcctccctcctcctcgccgcggccctttctgcctctgcggggctctggtcgccggccaaggaaaaacgaggctggaccctgaacagcgcgggctacctgctgggcccacatgccgttggcaaccacaggtcattcagcgacaagaatggcctcaccagcaagcgggagctgcggcccgaagatgacatgaaaccaggaagctttgacaggtccatacctgaaaacaatatcatgcgcacaatcattgagtttctgtctttcttgcatctcaaagaggccggtgccctcgaccgcctcctggatctccccgccgcagcctcctcagaagacatcgagcggtcctgagagcctcctgggcatgtttgtctgtgtgctgtaacctgaagtcaaaccttaagataatggataatcttcggccaatttatgcagagtcagccattcctgttctctttgccttgatgttgtgttgttatcatttaagattttttttttttggtaattattttgagtggcaaaataaagaatagcaattaaaaaaaaaaaaacaaaaaaaaaaaaaaaa

SEQ ID NO: 15 - DNA, 675 bp, Bos taurus
cttccgcgtccccgaggccgcgccatgcggtgagcgtccccggccctgccccgacccgactcgacggacgcgcggccccgccgacacaggacctgcagacaccccaggacccgcagacatcccccgaccctccgggccccgctcaagatgcccagaggctccgtcctgctgctcgcctccctgctcctcgcagcggccctttcagccaccctgggcctcgggtcaccggtgaaggagaagagaggctggaccctgaacagcgctgggtaccttctcggaccacatgcgctcgacagccacaggtcatttcaagacaagcatggcctcgccggcaagcgggaactcgagcctgaagacgaagcccggccaggaagctttgacagaccactggcggagaacaacgtcgtgcgcacgataatcgagtttctgactttcctgcatctcaaagacgccggcgccctggagcgcctgcccagtctccccacagcagagtccgcagaagacgccgagaggtcctgagcgggctcccgcgcgtcggtctccctgtgtcacgcgcagtcgtgctcccaggaggatgcccatcgcatggcaaccgccccatccccgctgccctgatgctgtgtccgtaccatttcaggtttttcccctttggtcataagtttcagtggcaaaatt

SEQ ID NO: 16 - DNA, 774 bp, Sus scrofa
acacgtcgaaggagcccggctgccgcgcttccctctctgtgtccccgaggccacgccatgcggtgagcgccctccagccctgcccgacccaaccggacccgcgtccccgccgacagcccaggacccgctggcacccggggaccccctggcatctcagacccgccgacccccggggcccgccgacaccccaagacccaccgacactccgggacccgccgtcgctcaagatgcccagaggctgcgccctcctgctggcctccctactcctcgcttcggccctttcagccaccctggggctcgggtcaccggtgaaggaaaagagaggctggactctgaacagcgctggctaccttcttgggccacatgccatcgacaaccacagatcattccacgacaagtatggccttgctggcaagcgggaactcgaacccgaagacgaagccaggccgggaggctttgaccggctgcagtcagaggacaaagccatacgcacgataatggagtttctggctttcttgcatctcaaagaggcgggggccctggggcgcctgcccggcctcccctcggcagcatcctcagaagacgcgggacagtcctgaggtggctccggcatcttcgtctcggcgttgtcgagctccgagacggtgacggtctcacgccagcgaaggcagcgtaaccacccctgtcgtccctgcccagtgctgtgttgctgtggtgtcagatcttcttcctttgggagtaggtttgagccgcaaaataaaaactgcagctgct

SEQ ID NO: 17 - DNA, 20 bp, Artificial Sequence
  Description of Artificial Sequence Synthetic primer
tgcagtaagcgaccatccag

SEQ ID NO: 18 - DNA, 20 bp, Artificial Sequence
  Description of Artificial Sequence Synthetic primer
agcacaggacacacgtgcac

SEQ ID NO: 19 - DNA, 20 bp, Artificial Sequence
  Description of Artificial Sequence Synthetic primer
cgccttcatctgcaagttta

SEQ ID NO: 20 - DNA, 18 bp, Artificial Sequence
  Description of Artificial Sequence Synthetic primer
caggacggtctgtgcagt

SEQ ID NO: 21 - DNA, 20 bp, Artificial Sequence
  Description of Artificial Sequence Synthetic primer
tgcctttccaggccaccatc

SEQ ID NO: 22 - DNA, 20 bp, Artificial Sequence
  Description of Artificial Sequence Synthetic primer
gcgtaagtggcacgcgtgag

SEQ ID NO: 23 - DNA, 23 bp, Artificial Sequence
  Description of Artificial Sequence Synthetic primer
cctggctctttggggctttcgtg

SEQ ID NO: 24 - DNA, 22 bp, Artificial Sequence
  Description of Artificial Sequence Synthetic primer
agcgcgtagagcgcggccactg

SEQ ID NO: 25 - DNA, 23 bp, Artificial Sequence
  Description of Artificial Sequence Synthetic primer
tgacatcaagaaggtggtgaagc

SEQ ID NO: 26 - DNA, 24 bp, Artificial Sequence
  Description of Artificial Sequence Synthetic primer
aaggtggaagagtgggagttgctg

SEQ ID NO: 27 - DNA, 20 bp, Artificial Sequence
  Description of Artificial Sequence Synthetic oligonucleotide
aatggccacgtagcgatcca

SEQ ID NO: 28 - DNA, 21 bp, Artificial Sequence
  Description of Artificial Sequence Synthetic oligonucleotide
gtagctgcaggctcaggttcc

SEQ ID NO: 29 - DNA, 22 bp, Artificial Sequence
  Description of Artificial Sequence Synthetic oligonucleotide
gtggccgtggtgagcctggcct

SEQ ID NO: 30 - protein, 7 aa, Artificial Sequence
  Description of Artificial Sequence Synthetic peptide
PPALALA

SEQ ID NO: 31 - protein, 20 aa, Artificial Sequence
  Description of Artificial Sequence Synthetic peptide
GWTLNSAGYLLGPQQFFGLM

(19) United States
(12) Patent Application Publication
(10) Pub. No.: US 2008/0132445 A1
(43) Pub. Date: Jun. 5, 2008

(54) Method for Inducing Mammary Epithelial Cell Differentiation

(75) Inventors: Christopher J. Ormandy, Lane Cove (AU); Matthew John Naylor, Greater Manchester (GB)

Correspondence Address: FOLEY & LARDNER LLP, P.O. BOX 80278, SAN DIEGO, CA, 92138-0278, US

(73) Assignee: Garvan Institute of Medical Reserach

(21) Appl. No.: 10/529,094

(22) PCT Filed: Sep. 25, 2003
(86) PCT No.: PCT/AU03/01266

§ 371 Date: Jan. 9, 2006

Related U.S. Application Data
(60) Provisional application No. 60/413,978, filed on Sep. 25, 2002.

(51) Int. Cl. A61K 38/00; C12N 5/06; A61K 31/56; A01K 67/00; A61P 43/00

(52) U.S. Cl. 514/12; 435/377; 514/2; 514/13; 514/14; 514/182; 800/13; 800/14; 800/15; 800/16; 800/17

(57) ABSTRACT

The present invention relates to compositions and methods for inducing mammary epithelial cell differentiation in mammalian subjects. More specifically, the present invention relates to methods for inducing mammary epithelial cell differentiation which comprise increasing the levels of galanin in the mammary tissue of the subject. In one aspect the present invention relates to a method of increasing milk production in a lactating mammal which comprises increasing the level of galanin or an analog thereof in the mammal. In another aspect the present invention relates to a method of enhancing mammary development in a mammal, the method comprising administering to the mammal galanin or an analog thereof in conjunction with prolactin or an analog thereof. In yet another aspect the present invention relates to a method for inhibiting mammary epithelial tumours by administering an inhibitorially effective therapeutic amount of galanin or an analog thereof.

### FIELD OF THE INVENTION

The present invention relates to compositions and methods for inducing mammary epithelial cell differentiation in mammalian subjects. More specifically, the present invention relates to methods for inducing mammary epithelial cell differentiation which comprise increasing the levels of galanin or a functional analog or agonist thereof in the mammary tissue of the subject. In one aspect the present invention relates to a method of increasing milk production in a lactating mammal which comprises increasing the level of galanin or an analog or agonist thereof in the mammal. In another aspect the present invention relates to a method of enhancing mammary development in a mammal, the method comprising administering to the mammal galanin or an analog thereof in conjunction with prolactin or an analog thereof. In yet another aspect the present invention relates to a method for inhibiting mammary epithelial tumours by administering an inhibitorially effective therapeutic amount of galanin or an analog thereof.

### BACKGROUND OF THE INVENTION

Development of the mammary gland occurs in defined stages connected to embryonic, prepubertal, and pubertal stages of development, as well as during pregnancy, lactation and involution in an adult female. The mammary gland consists of two cellular compartments, the epithelium and surrounding stroma. The epithelium is derived embryonically from ectoderm, and comprises: (i) a branched ductal system (ducts branching into ductules, and terminating in lobules comprising alveoli that consist of secretory epithelium, and surrounded by contractile myoepithelium) that mainly develops during puberty; and (ii) the lobuloalveolar compartment that develops during pregnancy. Receptors for estrogen, progesterone and prolactin, the Stat5 transcription factors, cyclinD1, and the family of activins and inhibins are required for the establishment of functional mammary tissue. Mammary development is reviewed in detail by Hennighausen and Robinson, Devel. Cell, 1, 1-20, 2001, which is incorporated herein in its entirety by way of reference.

The secretory epithelium of the ductal system undergoes functional differentiation during parturition. The secretory compartment arises from stem cells during each pregnancy, produces milk during lactation, and is fully remodelled after weaning of the young. This remodelling is accompanied by the loss of the entire secretory epithelium.

In normal mammary glands, proliferation and differentiation of the secretory mammary epithelium requires prolactin, a prolactin receptor (PrlR) and an operable Jak2/Stat5 signalling pathway (Ormandy et al., Genes Dev. 11, 167-178, 1997; Liu et al., Genes Dev. 11, 179-186, 1997). Briefly, binding of prolactin or placental lactogen to PrlR induces receptor dimerization, leading to tyrosine phosphorylation of PrlR by Jak2. Subsequently, the transcription factors Stat5a and Stat5b are recruited by their SH2 domains to the receptor where they are also phosphorylated by Jak2. This phosphorylation of the Stat5 transcription factors is believed to lead to a cascade of intracellular events leading to cell proliferation and differentiation. For example, mice deficient in one of both Stat5 transcription factors have arrested mammary gland development, including impaired alveolar proliferation and functional differentiation (Liu et al., Genes Devel. 11, 179-186, 1997; Liu et al., Cell. Growth Differ. 9, 795-803, 1998; Miyoshi et al., J. Cell. Biol., 2001; Teglund et al., Cell 93, 841-850, 1998). Additionally, the effects of prolactin on cell growth are synergistic with the effects of progesterone, which appears to act, in part, by increasing the level of PrlR.

The mitogenic action of prolactin is important in tumorigenesis. Breast cancer or mammary tumor, is the most common cancer diagnosed amongst women. About one in nine women will develop breast cancer in their lifetime, and about 200,000 new cases of breast cancer are diagnosed annually in the United States with a mortality rate of about 20-25%. In the treatment of breast cancer, there is considerable emphasis on detection and risk assessment, because early and accurate staging of breast cancer has a significant impact on survival. For example, breast cancer detected at an early stage (stage T0, discussed below) has a five-year survival rate of 92%. Conversely, if the cancer is not detected until a late stage (i.e., stage T4), the five-year survival rate is reduced to about 13%.

Breast cancers, or mammary gland tumors, may consist of lobular lesions, stromal lesions, ductal carcinoma (non-invasive ductal carcinoma or invasive ductal carcinoma), proliferative fibrocystic changes, or epitheliosis. Intraductal papilloma and/or atypical ductal hyperplasia are considered to be precursors to ductal carcinomas. Atypical ductal hyperplasia predicts a 4 fold increased relative risk for subsequent invasive ductal adenocarcinoma. As used herein, the term “breast cancer” shall be taken to include any one or more of these lesions, carcinomas or precursors, or a metastases thereof internal or external to the mammary gland.

Two current methods of breast cancer prevention involve prophylactic mastectomy (mastectomy performed before cancer diagnosis) and chemoprevention (chemotherapy before cancer diagnosis) which are drastic measures that limit their adoption even among women with increased risk of breast cancer. Administration of the prophylactic Tamoxifen has also been trialled and met with limited success. Accordingly, alternative methods of treating breast cancer are needed.

Approximately 20% of women, for one reason for another, have difficulty with breastfeeding. Part of this is due to fear of the procedure and part due to an inability of the child to stimulate the nipple adequately due, often, to abnormalities of the lip and the palet of the child. Difficulties with breast feeding may also come about as a result of a deficiency or health problem experience by the mother. For example, overweight women have more difficulty initiating breast feeding than normal women.

Difficulties are also often experience in breastfeeding where babies are born prematurely. Mothers of premature infants often cannot breastfeed because of a failure of lactation to be initiated. This failure is thought to be due to inadequate preparation of the pituitary gland for the secretion of prolactin.

Methods for enhancing lactation or augmenting milk production in lactating women are therefore desirable.

Galanin is a 29 amino acid peptide originally isolated from porcine intestine (Tatemoto et al, FEBS Lett 164:124-128, 1983) that has been implicated in the control of a number of biological processes including cognition, feeding behavior, neuroendocrine responses, mitogenesis and nociception (Iismaa and Shine, Results Probl. Cell Differ. 26:257-291, 1999). Galanin signals through a family of three G protein-coupled receptors, galanin receptors (Galr) 1-3 (Habert-Ortoli et al, Proc Natl Acad Sci USA, 91:9780-9783; Howard et al, FEBS Lett, 405:285-290, 1997; Wang et al, J. Biol. Chem., 272:31949-31952, 1997). The generation of mice carrying a loss-of-function mutation of the galanin gene has enabled investigation of the functions of galanin in vivo. Galanin regulates the development of sensory and cholinergic neurons, hippocampal excitability and modulation of the pain response. Overexpression of galanin in neurons suppresses epileptic-like induced seizures. Galanin is also a mitogen for the prolactin secreting pituitary lactotroph cells. Overexpression of galanin in the lactotroph induces hyperplasia and consequent hyperprolactinemia.

The galanin gene is located at chromosome 11q13, and like many genes in this region it is amplified in around 13% of breast cancers. Galanin is expressed by a number of breast cancer cell lines, but expression does not correlate with amplification. In contrast, galanin expression correlates with estrogen and progesterone receptor expression and is regulated by estradiol and progesterone (Ormandy et al, Cancer Res. 58:1353-1357). In the rat, serum levels of galanin increase during pregnancy and peak at mid pregnancy with levels seven fold greater then those observed in nulliparous animals (Vrontakis et al, Endocrinology 130:458-464).

To date, three subtypes of galanin receptors, referred to as GalR1, GalR2 and GalR3, have been cloned from several species (human, rat, mouse). Each receptor subtype has a high sequence homology between different species, but within a species the sequence similarities between different receptor subtypes are lower. For example, recombinant GalR1 (rGalR1) has only 40% amino acid identity to recombinant GalR2 (rGalR2), while, recombinant GalR3 (rGalR3) has 36% and 58% identity towards rGalR1 and RGalR2, respectively.

GalR1 is localised mainly in the hypothalamus, the hippocampus and the spinal cord, and seems to be negatively coupled to adenylyl cyclase through Gᵢ/Gₒ proteins. GalR1 needs the N-terminus of galanin for recognition. GalR1 has been cloned from rat hypothalamus, rat dorsal root ganglia, human placenta, human DNA library and from mouse brain. The main effector of GalR2 is phospholipase C mediated via G_{q/11}. It is activated by galanin(2-29) and [D-trp²]-galanin and has been shown to couple to inositol phospholipid hydrolysis. GalR3 was cloned from rat hypothalamus and is localised mainly in heart, spleen and testis and recognises galanin(2-29) as a specific ligand. GalR3 couples to Gᵢ/Gₒ proteins and mediates opening of G protein-coupled inward-rectifying potassium channels (GIRK).

Galanin-like peptide, GALP, is a recently described 60 amino acid neuropeptide isolated from porcine hypothalamus, that binds to GalR1 and GalR2 (Ohtaki et al, J. Biol. Chem. 274:37041-37045, 1999). GALP contains the non-variable 13 amino acids of galanin between positions 9 and 21, and represents an alternative endogenous ligand to galanin receptors.

### SUMMARY OF THE INVENTION

In work leading up to the present invention, the inventors observed for the first time that galanin acts directly on the mammary gland via the JAK/STAT pathway to induce epithelial differentiation. Galanin treatment of mammary gland tissue caused sustained activation of the STAT5 pathway and cell differentiation as measured by milk protein expression. This finding establishes a new role for galanin as a systemic hormone that controls mammary gland development and is useful in increasing milk production in lactating mammals.

Importantly, the inventors have also found that galanin exerts a differentiative activity without exerting proliferative activity in the mammary gland. More specifically, galanin produced induction of milk protein synthesis but failed to induce lobuloalveolar development. This contrasts to prolactin which exerts both proliferative and differentiative action. The finding that galanin forces dimmunition of proliferation and induces cell differentiation is an indication that galanin acts as a mammary tumour suppressor.

Accordingly, in a first aspect the present invention provides a method of inducing differentiation of mammary epithelial cells, the method comprising administering an effective amount of galanin or a functional analog or agonist thereof to the mammary epithelial cells.

In a further aspect the present invention provides a method of inducing differentiation of mammary epithelial cells in a mammal, the method comprising increasing the level of galanin or a functional analog or agonist thereof in the mammary tissue of the mammal.

In a further aspect the present invention provides a method of increasing milk production in a mammal, the method comprising increasing the level of galanin or a functional analog or agonist thereof in the mammary tissue of the mammal.

It will be appreciated that the methods of the present invention can be used to augment milk production in lactating mammals. The methods of the present invention can also be used, in conjunction with appropriate hormonal treatment (such as administration of estrogens, progesteron and oxytocin), to induce lactation in nonpregnant or nonlactating mammals,

Those skilled in the art will appreciated that the term “mammal” includes, without being limited to, primates (e.g., humans), cows, sheep, goats, horses, dogs, cats, rabbits, guinea pigs, rats, mice or other bovine, ovine, equine, canine, feline, rodent or murine species.

In the context of the present invention, the level of galanin in the mammary tissue of a mammal can be increased in any one of a number of different ways.

In a preferred embodiment, the level of galanin is increased by administering to a mammal an amount of galanin or a functional analog or agonist thereof effective to induce differentiation of mammary epithelial cells and/or increase milk production in the mammal.

In a preferred embodiment, the galanin analog is a polypeptide comprising the following fragment: GWTLNSAGYLLGP (SEQ ID NO:1).

In another embodiment the galanin is a human galanin polypeptide having the following amino acid sequence: GWTLNSAGYLLGPHAVGNHRSFSDKNGLTS (SEQ ID NO:2) or a functional equivalent thereof or a functional fragment thereof.

In another preferred embodiment the galanin is a bovine galanin polypeptide having the following amino acid sequence: GWTLNSAGYLLGPHALDSHRSFQDKHGLA (SEQ ID NO:3) or a functional equivalent thereof or a functional fragment thereof.

In another embodiment the galanin is a porcine galanin polypeptide having the following amino acid sequence: GWTLNSAGYLLGPHAIDNHRSFHDKYGLA (SEQ ID NO:4) or a functional equivalent thereof or a functional fragment thereof.

In another embodiment the galanin is a rat galanin polypeptide having the following amino acid sequence: GWTLNSAGYLLGPHAIDNHRSFSDKHGLT (SEQ ID NO:5) or a functional equivalent thereof or a functional fragment thereof.

In another embodiment the galanin has the following amino the amino acid sequence: GWTLNSAGYLLGPHAVNHRSFSDKNGLTS (SEQ ID NO:6) or a functional equivalent thereof or a functional fragment thereof.

In another embodiment the galanin analog is a human GALP (1-60) polypeptide having the following amino acid sequence: APAHRGRGGWTLNSAGYLLGPVLHLPQMGDQDGKRETALEILDLWKAIDGLP YSHPPQPS (SEQ ID NO:11) or a functional equivalent thereof or a functional fragment thereof.

In another embodiment the galanin analog is a porcine GALP (1-60) polypeptide having the following amino acid sequence: APVHRGRGGWTLNSAGYLLGPVLHPPSRAEGGGKGKTALGILDLWKAIDGLP YPQSQLAS (SEQ ID NO:12) or a functional equivalent thereof or a functional fragment thereof.

In another embodiment the galanin analog is a rat GALP (1-60) polypeptide having the following amino acid sequence: APAHRGRGGWTLNSAGYLLGPVLHLSSKANGGRKTDSALEILDLWKAIDGLR YSRSPRMT (SEQ ID NO:13) or a functional equivalent thereof or a functional fragment thereof.

In another embodiment the galanin analog is selected from the group consisting of:

(i) Galanin-(2-29) (i.e. deletion of first amino acid);
(ii) Galanin-3-29) (i.e. deletion of first 2 amino acids);
(iii) Galanin-(1-15) (i.e. deletion of amino acids 16-29/30);
(iv) Galanin-(1-16) (ie. deletion of amino acids 17-29/30);
(v) M40: galanin-(1-13)-Pro-Pro-Ala-Leu-Ala-Leu-Ala-amide;
(vi) M15 (galantide): Gly-Trp-Thr-Leu-Asn-Ser-Ala-Gly-Tyr-Leu-Leu-Gly-Pro-Gln-Gln-Phe-Phe-Gly-Leu-Met-NH₂ (SEQ ID NO: 13);
(vii) M35: galanin (1-13)-bradykinin (2-9) amide;
(viii) M32: galanin (1-13)-neuropeptide Y(25-36) amide; and
(ix) C7: galanin(1-13)-spantide amide.

The present inventors have shown that all three galanin receptors (GalR1, GalR2 and GalR3) are expressed in the mammary gland during pregnancy. GalR2 expression was detected most strongly during lactation and involution.

Accordingly, in aspects of the invention that relate to increased milk production, it is preferred that the galanin analog is an agonist of the GalR2 receptor. It is known that the GALP (1-60) polypeptide binds GalR2 with similar affinity to galanin but has poor affinity for GalR1. Thus, in light of its GalR2 specificity, the GALP(1-60) polypeptide is a preferred galanin analog for use in increasing milk production in a mammal. Galanin(2-16) is also a preferred analog for use in increasing milk production in a mammal.

Other methods for increasing the level of galanin in the mammary tissue of a mammal are clearly encompassed by the present invention. For example, it is known that the galanin promoter contains an estrogen response element and that estrogen can increase galanin expression about 4000 fold. See, for example, Howard et al, 1997, FEBS Lett. 405:285-290; and Kaplan et al, 1988, Proc. Natl. Acad. Sci. 85:1065-1069.

Accordingly, in a further embodiment of the present invention, the level of galanin in the mammary tissue is increased by administering to the mammal an amount of estrogen or a functional analog thereof effective to increase expression of galanin in the mammal. In one embodiment, the estrogen analog is estradiol. Other analogs that function as estrogen agonists and are therefore suitable for use in the present invention are disclosed in U.S. Pat. No. 6,441,193 and U.S. Pat. No. 6,274,618.

In order to increase the level of galanin in the mammary tissue of the mammal, it is preferred that the estrogen is administered orally or parenterally to the mammal. Preferably, the estrogen concentration ranges from about 0.5 to about 2.0 mg/kg body weight.

In another embodiment the level of galanin is increased by gene therapy. For example, the level of expression of galanin in the mammary tissue may be increased by modifying a control element, such as the promoter, of the galanin gene in tissue cells of the mammal. Alternatively, the level of galanin in the mammary tissue may be increased by administering to the mammal a recombinant construct capable of expressing a galanin polypeptide or an analog thereof. The construct may comprise a promoter that specifically targets expression of galanin in the mammary tissue, although tissue specific expression is not essential. For example, over-expression of galanin in another tissue, such as the pituitary or placenta, which results in increased levels of galanin circulating in the serum of the mammal is also encompassed by the present invention.

The present inventors have also observed synergistic modes of interaction between galanin and prolactin. For example, when galanin is used in combination with prolactin in mammary organ culture, larger and more numerous lobules were produced than with prolactin alone.

Accordingly, in aspects of the invention that relate to increased milk production, it is preferred that the increase in the level or activity of galanin or a functional analog or agonist thereof is brought about in conjunction with an increase in level or activity of prolactin or an analog thereof. For example, galanin or an analog or agonist thereof may be administered to the mammal in conjunction with prolactin or an analog thereof.

In a further aspect the present invention provides a method of enhancing mammary development in a mammal, the method comprising increasing the level of galanin or a functional analog or agonist thereof and increasing the level of prolactin or an analog thereof.

The level of prolactin may be increased by any suitable method. For example, the level of prolactin may be increased by administration of a prolactin stimulator. Exemplary of prolactin stimulators are dopamine antagonists, i.e. metoclopramide, haloperidol, pimozide, phenothiazine, sulpiride, chlorpromazine and serotonin agonists, i.e. MAO inhibitors, e.g. pargyline. synthetic morphine analogs, e.g. methadone, antiemetics, e.g. metoclopramide, antipsychotics, e.g. sulpiride, estrogens and others, e.g. tryptophan and 5-hydroxy-tryptophan.

In a preferred embodiment of this aspect, the method comprises administering galanin or an analog or agonist thereof in conjunction with prolactin or an analog thereof. The galanin or analog thereof and prolactin or analog thereof may be administered sequentially or simultaneously to the mammal.

Examples of prolactin analogs are described in Goffin et al, J. Biol. Chem., 271:16573-16579, 1996; Goffin et al, Mol. Endocrinol., 6:1381-1392, 1992; Kinet et al, J. Biol. Chem., 271:14353-14360, 1996; and Bernichtein et al, J. Biol. Chem. 2003 278:35988-99, 2003.

It will be appreciated that the methods of the present invention are useful for augmenting or enhancing lactation in women in need thereof. These methods are particularly useful, for example, in cases where lactation has not been initiated, for example with premature births, or where the child is unable to stimulate the nipple to induce adequate levels of lactation.

It will also be appreciated that the methods of the present invention have particular application to the dairy industry. In particular, the methods of the present invention can be used to augment or enhance lactation in livestock such as cows, goats and sheep.

In the context of enhancing lactation or milk production in livestock, the level of galanin in the mammal may be increased via production of transgenic animals. This may be achieved, for example, by producing a transgenic mammal having integrated in its genome a nucleic acid construct comprising a sequence encoding galanin or an analog thereof, wherein the transgenic mammal expresses galanin or an analog thereof at an elevated level compared to an equivalent non-transgenic mammal.

By “equivalent non-transgenic mammal” we mean a mammal which has substantially the same genome as the transgenic mammal except that it lacks the nucleic acid construct comprising a sequence encoding galanin or an analog thereof.

In one embodiment of this aspect, the sequence encoding galanin is selected from a cDNA sequence as shown in SEQ ID NO:14 (which encodes human galanin) or fragment thereof, SEQ ID NO:15 (which encodes bovine galanin) or a fragment thereof and SEQ ID NO:16 (which encodes porcine galanin) or a fragment thereof.

In a preferred embodiment of this aspect the nucleic acid construct further comprises a mammary specific promoter operably linked to the sequence encoding galanin or an analog thereof. Preferably, the mammary specific promoter is selected from the group consisting of the WAP promoter, the murine mammary tumour virus (MMTV) long terminal repeat, the neu-related lipocalin (NRL) promoter, the beta-casein promoter, the beta-lactoglobulin (BLG) promoter and the beta 1,4 galactosyltransferase promoter.

In another aspect the present invention provides a transgenic mammal having integrated in its genome a nucleic acid construct comprising a sequence encoding galanin or an analog thereof, wherein the transgenic mammal expresses galanin or an analog thereof at an elevated level compared to an equivalent non-transgenic mammal, and wherein the level of milk production is increased in the transgenic mammal when compared to an equivalent non-transgenic mammal.

In a preferred embodiment of this aspect the transgenic mammal is a cow, sheep, pig or goat.

In yet another aspect the present invention provides a method of inhibiting the growth of a mammary epithelial tumour in a subject, the method comprising administering to the subject an inhibitorially effective therapeutic amount of galanin or a functional analog thereof.

In one embodiment of this aspect of the invention the epithelial tumour is a naturally occurring epithelial tumour where there is no apparent carcinogenic etiologic agent.

In a further aspect the present invention provides a method for the treatment of a mammary hyperproliferative disease in a subject, the method comprising administering to the subject an inhibitorially effective therapeutic amount of galanin or a functional analog thereof.

In a preferred embodiment of this aspect of the invention the mammary hyperproliferative disease is cancer. Those skilled in the art will be aware that as a carcinoma progresses, metastases occur in organs and tissues outside the site of the primary tumor. For example, in the case of mammary cancer, metastases commonly appear in a tissue selected from the group consisting of omentum, cervical tissue, abdominal fluid, lymph nodes, lung, liver, brain, and bone. Accordingly, the term “mammary cancer” as used herein shall be taken to include an early or developed tumor of the mammary gland and any metastases outside the mammary gland that occurs in a subject having a primary tumor of the breast.

### BRIEF DESCRIPTION OF THE DRAWINGS

FIG. 1: Mammary gland development and differentiation in Galanin knockout mice treated with prolactin. A, carmine stained whole mounts of 4th mammary glands of Gal+/+ and Gal−/− at day 12 of pregnancy, note reduced alveolar density in Gal−/− glands. B, whole mounts taken on the 1st day postpartum, with and without prolactin treatment, note increase in alveolar density with prolactin treatment. C, haematoxylin and eosin stained 5 μm sections from mammary glands at 1st day post-partum, note retention of pink-staining proteinacious secretions and oil droplets in Gal−/− glands that are absent in Gal+/+glands. Prolactin treated Gal−/− glands show both retention and loss of the pink-staining proteinaceous secretions and oil droplets. D, lactation in Gal+/+, Gal−/− and Gal−/− mice treated with PRL throughout pregnancy. Loss of Gal prevented the first lactation. Prolactin treatment prevented lactational failure of Gal−/− mice. E and F, milk protein (WDMN-1, β-casein and WAP) and keratin 18 mRNA expression by quantitative RT-PCR at the 1st day post-partum. Fold difference in expression levels expressed as Gal−/− verses Gal+/+ (E) and Gal−/− treated with PRL verse Gal+/+ (F). Prolactin treatment failed to rescue the loss of milk protein expression caused by the knockout of Gal.

FIG. 2: Galanin and galanin receptor expression in the mammary gland. Expression of galanin and galanin receptors at various developmental stages of mammary gland development by RT-PCR and hybridization of RT-PCR products with an internal oligonucleotide (Galr1-3). Developmental stages are virgin mice at estrous (est.), virgin mice at diestrous (diest.), days 7, 12 and 16 of pregnancy (7D, 12D & 16D pregnant), lactation and 5 days of involution (5D invol.).

FIG. 3: Transplant of Gal−/− epithelium or stroma to Rag1−/− hosts. Carmine stained whole mounts of Gal−/− (A,C) & Gal+/+ (B,D) epithelium transplanted into the fat pad of Rag1−/− mice cleared of endogenous epithelium. (A,B) virgin (C,D) 1st day post-partum. Inserts haematoxylin and eosin stained 5 μm sections from the same glands. Deletion of galanin gene from the epithelium does not effect normal mammary gland morphology or histology. Other genotype and tissue recombinations produced identical results (not shown).

FIG. 4: Galanin acts directly on the mammary gland to induce lobuloalveoli development. Whole mounts of mammary glands following whole organ culture in vitro after culture in the presence of insulin, aldosterone and hydrocortisone (IAH), with or without galanin and PRL as indicated. Arrows indicate lobuloalveoli. (H&E) haematoxylin and eosin stained 5 μm sections from the same glands. Western blot analysis of the expression of milk proteins, STAT5, ERK and Akt in mammary glands following IAH, + galanin and/or PRL treatment. Milk protein (α-casein, β-casein and WAP) expression in explant mammary glands demonstrates that milk protein levels are increased following galanin+PRL, PRL or galanin treatment alone. Increased levels of phosphorylated STAT5 was observed in mammary glands following treatment with galanin and/or PRL. Galanin alone was not able to induce activation of the MAP kinase pathway. Phosphorylated ERK1/2 was increased in mammary glands treated with PRL or PRL+galanin despite a decrease in the total levels of ERK. This demonstrates marked specific activation of MAP kinase signaling in those glands treated with PRL. Examination of the PI3 kinase pathway revealed decreased mobility but no increase in total Akt in explants receiving PRL. This decrease in mobility was not due to phosphorylation of the two residues most commonly associated with Akt activation.

FIG. 5: Transcriptional interaction between galanin and prolactin revealed by transcript profiling. Transcript profiling of the cultured mammary glands detailed in FIG. 4 using Affmetrix U74A2 chips. Principle components analysis with genes coloured according to MAS5 calls of increasing (green) or decreasing (red) gene expression in response to treatment with galanin (G), PRL (P), or galanin+PRL (PG), compared to IAH alone. Groups correspond to the sets shown in FIG. 6A and the identity of set members is shown in FIG. 6B. Galanin treatment induces transcriptional changes that are also induced by prolactin (i). A set of mainly increasing transcriptional changes was identified that requires both prolactin and galanin (ii). Prolactin's transcriptional activity independent of galanin was robust (iii), but in contrast galanin's activity independent of prolactin was virtually non-existent (iv). The reason for this is demonstrated in (v), where clear antagonism of galanin's transcriptional effects by prolactin was seen. In contrast galanin antagonised only a small proportion of prolactin induced transcriptional changes (vi).

FIG. 6: Identity of the genes that are regulated by prolactin and galanin. A, Venn diagram showing the total number of genes found to be increasing or decreasing at least 1.7 fold in response to treatment of mammary explants with galanin, PRL, or galanin+PRL, in comparison to IAH only. These sets correspond to the principle components analysis shown in FIG. 5. B, selected genes identified by the Venn diagram approach shown in FIG. 6A. Labels and colours indicate their position in the Venn diagram. Fold change determined using MAS5 and selected candidates verified using quantitative RT-PCR.

FIG. 7: Summary of the endocrine role of galanin in mammary gland development. The stages of mammary gland development are shown schematically with causative reproductive events indicated above and descriptions of subsequent morphological changes given above each dashed arrow. Hormone secretion is shown by solid arrows. Regulatory influences on hormones or morphology are indicated by dashed lines that are positive (arrow heads) or negative (lines).

### DETAILED DESCRIPTION OF THE INVENTION

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Each embodiment described is to be applied mutatis mutandis to each and every other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A. Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. Land Fenichel, R. L. (1976). Biochem. Biophys. Res. Commun. 73 336-342
11. Merrifield, R. B. (1963). J. Am. Chem. Soc. 85 2149-2154.
12. Barany, G. and Merrifield, R. B. (1979) in The Peptides (Gross, E. and Meienhofer, J. eds.), vol. 2, pp. 1-284, Academic Press, New York.
13. Wiinsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
17. Handbook of Experimental Immunology, Vols. I-IV (D. M. Weir and C. C. Blackwell, eds., 1986, Blackwell Scientific Publications).

### Galanin and Analogs Thereof.

As used herein the term “galanin” embraces all known galanins including, for example, human, rat, murine and porcine galanin.

Although first isolated from porcine intestine, galanin is widely distributed in the central and peripheral nervous system. Galanin in most species is a 29 amino acid peptide with an amidated carboxyl terminus. Human galanin is unique in that it is longer, 30 amino acids, and is not amidated. There is strong conservation of the galanin sequence with the amino terminal fourteen residues being conserved in all species (with the exception of tuna in which Ser at residue 6 is replaced with Ala).

Accordingly, in a preferred embodiment of the present invention the galanin polypeptide comprises the following fragment: GWTLNSAGYLLGP (SEQ ID NO:1).

In a further preferred embodiment the galanin is a human galanin polypeptide having the following amino acid sequence: GWTLNSAGYLLGPHAVGNHRSFSDKNGLTS (SEQ ID NO:2) or a functional equivalent thereof or a functional fragment thereof.

In another embodiment the galanin is a bovine galanin polypeptide having the following amino acid sequence: GWTLNSAGYLLGPHALDSHRSFQDKHGLA (SEQ ID NO:3) or a functional equivalent thereof or a functional fragment thereof.

In another embodiment the galanin is a porcine galanin polypeptide having the following amino acid sequence: GWTLNSAGYLLGPHAIDNHRSFHDKYGLA (SEQ ID NO:4) or a functional equivalent thereof or a functional fragment thereof.

In another embodiment the galanin is a rat galanin polypeptide having the following amino acid sequence: GWTLNSAGYLLGPHAIDNHRSFSDKHGLT (SEQ ID NO:5) or a functional equivalent thereof or a functional fragment thereof.

In another embodiment the galanin has the following amino the amino acid sequence: GWTLNSAGYLLGPHAVNHRSFSDKNGLTS (SEQ ID NO:6) or a functional equivalent thereof or a functional fragment thereof.

In another embodiment the galanin is in the form of a human precursor polypeptide having the following amino acid sequence: MARGSALLLASLLLAAALSASAGLWSPAKEKRGWTLNSAGYLLGPHAVGNHR SFSDKNGLTSKRELRPEDDMKPGSFDRSIPENNIMRTIIEFLSFLHLKEAGALDR LLDLPAAASSEDIERS(SEQ ID NO:7) or a functional equivalent thereof or a functional fragment thereof.

In another embodiment the galanin is in the form of a bovine precursor polypeptide having the following amino acid sequence: MPRGSVLLLASLLLAAALSATLGLGSPVKEKRGWTLNSAGYLLGPHALDSHRS FQDKHGLAGKRELEPEDEARPGSFDRPLAENNVVRTIIEFLTFLHLKDAGALER LPSLPTAESAEDAERS (SEQ ID NO:8) or a functional equivalent thereof or a functional fragment thereof.

In another embodiment the galanin is in the form of a porcine precursor polypeptide having the following amino acid sequence: MPRGCALLLASLLLASALSATLGLGSPVKEKRGWTLNSAGYLLGPHAIDNHRS FHDKYGLAGKRELEPEDEARPGGFDRLQSEDKAIRTIMEFLAFLHLKEAGALG RLPGLPSAASSEDAGQS (SEQ ID NO:9) or a functional equivalent thereof or a functional fragment thereof.

In another embodiment the galanin is in the form of a human GALP precursor polypeptide having the following amino acid sequence: MAPPSVPLVLLLVLLLSLAETPASAPAHRGRGGWTLNSAGYLLGPVLHLPQMG DQDGKRETALEILDLWKAIDGLPYSHPPQPSKRNVTFAKPEIGDLGMLSMKI PKEEDVLKS (SEQ ID NO:10) or a functional equivalent thereof or a functional fragment thereof.

In another embodiment the galanin is a human GALP (1-60) polypeptide having the following amino acid sequence: APAHRGRGGWTLNSAGYLLGPVLHLPQMGDQDGKRETALEILDLWKAIDGLP YSHPPQPS (SEQ ID NO:11) or a functional equivalent thereof or a functional fragment thereof.

In another embodiment the galanin is a porcine GALP (1-60) polypeptide having the following amino acid sequence: APVHRGRGGWTLNSAGYLLGPVLHPPSRAEGGGKGKTALGILDLWKAIDGLP YPQSQLAS (SEQ ID NO:12) or a functional equivalent thereof or a functional fragment thereof.

In another embodiment the galanin is a rat GALP (1-60) polypeptide having the following amino acid sequence: APAHRGRGGWTLNSAGYLLGPVLHLSSKANGGRKTDSALEILDLWKAIDGLR YSRSPRMT (SEQ ID NO:13) or a functional equivalent thereof or a functional fragment thereof.

As used herein in relation to polypeptide sequences the term “functional equivalent” is intended to cover minor variations in the amino acid sequence which do not deleteriously affect the biological activity of the polypeptide. It will be recognised by those skilled in the art that a number of modifications may be made to the peptide of the present invention without deleteriously affecting the biological activity of the peptide. This may be achieved by various changes, such as insertions, deletions and substitutions, either conservative or non-conservative in the peptide sequence where such changes do not substantially decrease the biological activity of the peptide. By conservative substitutions the intended combinations are:

### G,A; V,I,L,M; D,E; N,Q; S,T; K,R,H;

and

### F,Y,W.

It may also be possible to add various groups to the peptide of the present invention to confer advantages such as increased potency or extended half-life in vivo without substantially decreasing the biological activity of the peptide. These additions and changes include the introduction of D-amino acid residues and the formation of cyclic analogues.

A “functional fragment” retains at least 10%, more preferably at least 25%, more preferably at least 50%, more preferably at least substantially the same biological activity as that of the full length polypeptide.

It is preferred that galanin analogs have at least 10%, more preferably at least 25%, more preferably at least 50%, more preferably at least substantially the same biological activity of any one of the galanin polypeptides shown in SEQ ID NOs 1-13.

In another embodiment the galanin analog is selected from the group consisting of:

(i) Galanin-(2-29) (i.e. deletion of first amino acid);
(ii) Galanin-3-29) (i.e. deletion of first 2 amino acids);
(iii) Galanin-(1-15) (i.e. deletion of amino acids 16-29/30);
(iv) Galanin-(1-16) (ie. deletion of amino acids 17-29/30);
(v) M40: galanin-(1-13)-Pro-Pro-Ala-Leu-Ala-Leu-Ala-amide;
(vi) M15 (galantide): Gly-Trp-Thr-Leu-Asn-Ser-Ala-Gly-Tyr-Leu-Leu-Gly-Pro-Gln-Gln-Phe-Phe-Gly-Leu-Met-NH₂ (SEQ ID NO: 13);
(vii) M35: galanin (1-13)-bradykinin (2-9) amide;
(viii) M32: galanin (1-13)-neuropeptide Y(25-36) amide; and
(ix) C7: galanin(1-13)-spantide amide.

Preferred galanin analogs include the GalR1 agonist galanin (1-16), the GalR2 agonists galanin (2-16) and GALP, and the GalR3 agonist galanin (2-29).

These analogues and others may be generated by several means known to those skilled in the art, such as, for example:
(i) digestion of a galanin polypeptide or an immunologically active derivative thereof or a functional equivalent thereof, using a reagent such as, for example, cyanogen bromide, S-ethyltrifluorothioacetate, trypsin, chymotrypsin, pepsin, or thermolysin;
(ii) chemical peptide synthesis of a galanin polypeptide or a peptide derived therefrom that comprises at least about 5-10 amino acids in length, or a functionally equivalent peptide thereto (eg., a mimotope), using art-recognized techniques, such as, for example, Fmoc chemistry (reviewed by Fields (ed), Methods. Enzymol. 289, Academic Press, 1997 (whole of volume); Hecht, S. M. (ed) Bioorganic Chemistry: Peptides and Proteins, Oxford University Press: New York, ISBN 0-19-508468-3, 1998; Mayo, TIBTECH 18, 212-217, 2000);
(iii) by recombinant expression of a nucleic acid fragment of a full-length galanin protein-encoding region, or an equivalent thereof in a suitable cellular or cell-free expression system (see below); and
(iv) subjecting the nucleotide sequence of a full-length galanin protein-encoding region or a functional equivalent thereof to site-directed mutagenesis (reviewed by Hecht, S. M. (ed) Bioorganic Chemistry: Peptides and Proteins, Oxford University Press: New York, ISBN 0-19-508468-3, 1998), so as to produce single or multiple nucleotide substitutions, deletions and/or insertions that have minimal adverse effect on the antigenicity of the peptide encoded by the mutated sequence relative to the wild-type (non-mutant) sequence or the ability of said peptide to bind antibodies that recognize the full-length native galanin polypeptide.

For the purpose of producing derivatives using standard peptide synthesis techniques, such as, for example, Fmoc chemistry, a length not exceeding about 30-50 amino acids in length is preferred, as longer peptides are difficult to produce at high efficiency. Longer peptide fragments are readily achieved using recombinant DNA techniques wherein the peptide is expressed in a cell-free or cellular expression system comprising nucleic acid encoding the desired peptide fragment.

For producing galanin polypeptides or analogs thereof by recombinant means, a galanin protein-encoding region is placed in operable connection with a promoter or other regulatory sequence capable of regulating expression in a cell-free system or cellular system.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. Furthermore, the regulatory elements comprising a promoter are usually positioned within 2 kb of the start site of transcription of the gene. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning. A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others.

A wide range of additional host/vector systems suitable for expressing galanin polypeptides or immunological derivatives thereof are available publicly, and described, for example, in Sambrook et al (In: Molecular cloning. A laboratory manual, second edition, Cold Spring Harbor Laboratory, Cold Spring Harbor, N.Y., 1989).

### Screening for Galanin Agonists

When used herein the term “galanin agonist” refers to a compound that binds to a galanin receptor and produces a cellular response that is at least about equivalent to that of galanin, and that may be greater than that of galanin.

As mentioned above, three subtypes of galanin receptors, referred to as GalR1, GalR2 and GalR3, have been cloned to date. The GalR1 DNA sequence is described in Habert-Ortoli et al, Proc Natl. Acad. Sci., USA, 91:9780-9783, 1994. The GalR2 DNA sequence is described in U.S. Pat. No. 6,544,753. The GalR3 DNA sequence is described in U.S. Pat. No. 6,511,827.

These receptors may be employed in screening assays for identifying suitable galanin agonists for use in the present invention.

Screening assays which are suitable for this purpose include binding assays (competition for ¹²⁵I-galanin binding), coupling assays (including galanin-mediated inhibition of forskolin-stimulated adenylate cyclase in cells expressing galanin receptors), measurement of galanin-stimulated calcium release in cells expressing galanin receptors (such as aequorin assays), stimulation of inward rectifying potassium channels (GIRK channels, measured by voltage changes) in cells expressing galanin receptors, and measurement of pH changes upon galanin stimulation of cells expressing galanin receptors as measured with a microphysiometer.

Host cells may be cultured under suitable conditions to produce a galanin receptor. An expression vector containing DNA encoding the receptor may be used for expression of receptor in a recombinant host cell. Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including but not limited to Drosophila, Spodoptera, and silkworm derived cell lines. Cell lines derived from mammalian species which are suitable and which are commercially available include, but are not limited to, L cells L-M(TK-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

The specificity of binding of compounds showing affinity for the receptor is shown by measuring the affinity of the compounds for cells transfected with the cloned receptor or for membranes from these cells. Expression of the cloned receptor and screening for compounds that inhibit the binding of radiolabeled ligand to these cells provides a rational way for rapid selection of compounds with high affinity for the receptor. These compounds identified by the above assays may be agonists of the receptor and may be peptides, proteins, or non-proteinaceous organic molecules. Alternatively, functional assays of the receptor may be used to screen for compounds which affect the activity of the receptor. Such functional assays range from ex vivo muscle contraction assays to assays which determine second messenger levels in cells expressing the receptor. The second messenger assays include, but are not limited to, assays to measure cyclic AMP or calcium levels or assays to measure adenyl cyclase activity. These compounds identified by the above assays may be agonists of the receptor. The functional activity of these compounds is best assessed by using the receptor either natively expressed in tissues or cloned and exogenously expressed.

One assay which is particularly suitable for identifying galanin agonists comprises: a) culturing cells expressing a galanin receptor in the presence of a candidate compound and b) measuring the galanin receptor activity or second messenger activity. If desired, the determined activity can be compared to a standard, such as that measured using galanin as the compound. In preferred embodiments, the cells are transformed and express the GalR2 receptor.

### Modes of Administration

A variety of routes of administration are possible including, but not necessarily limited to oral, dietary, topical, parenteral (e.g., intravenous, intraarterial, intramuscular, subcutaneous injection), and inhalation (e.g., intrabronchial, intranasal or oral inhalation, intranasal drops) routes of administration, depending on the agent and disease or condition to be treated. For respiratory allergic diseases such as asthma, inhalation is a preferred mode of administration.

Formulation of an agent to be administered will vary according to the route of administration selected (e.g., solution, emulsion, capsule). An appropriate composition comprising the agent to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

It will also be appreciated that in the context of the present invention, the galanin or analog thereof can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The mouse whey acidic protein (WAP) gene promoter of about 1 kbp in length and/or having functional elements required for expression in mammary tissue is particularly preferred for this purpose. The WAP gene is expressed almost exclusively in mammary tissue (Pittius et al., Proc Natl Acad Sci USA 85, 5874-5878, 1988), and its transcription is induced several thousand-fold at mid-pregnancy and remains high throughout lactation (Pittius et al., Mol Endocrinol 2, 1027-1032, 1988). Induction and maintenance of WAP gene expression is mediated to a large extent through the prolactin and glucocorticoid signalling pathways. The distal Stat5-binding site of the WAP promoter is required for high level and prolactin-modulated (i.e. prolactin-induced) expression (Li and Rosen, Mol Cell Biol 15, 2063-2070, 1995). The distal NF1 site also appears to be required for WAP gene expression (Li and Rosen, Mol Cell Biol 15, 2063-2070, 1995), and the promoter proximal Ets site mediates transcription in late pregnancy but not for high expression throughout lactation (McKnight et al., Mol Endocrinology 9, 717-724, 1995). Elements that confer glucocorticoid responsiveness on the WAP promoter have also been mapped in the promoter distal region. Binding sites for transcription factors belonging to the NF1 and Ets family have been located within 200 bp of the transcriptional start site.

Other suitable promoters for expression in the mammary tissue include the B-lactoglobulin (BLG) promoter which is described in U.S. Pat. No. 5,322,775; the neu-related lipocalin (NRL) promoter as described in Rose-Hellekant et al, Oncogene, 22:4664-4674, 2003; the beta-casein promoter as described in Altiok et al, Mol. Cell. Biol., 13:7303-7319, 1993; the beta 1,4-galactosyltransferase promoter (described in Charron et al, Proc. Natl. Acad. Sci. USA, 95:14805-14810, 1998); and the murine mammary tumour virus long terminal repeat (Sinn et al., Cell, 49, 465, 1987).

Preferred vectors for expression in mammalian cells (eg. 293, COS, CHO, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of galanin or an analog thereof in 293T cells, wherein the expressed peptide or protein can be purified free of non-specific proteins, using standard affinity techniques that employ a Nickel column to bind the protein via the His tag.

In the context of the present invention it is contemplated that cells can be engineered to express galanin or an analog thereof by gene therapy methods. For example, DNA encoding galanin, or an active fragment or derivative thereof, can be introduced into an expression vector, such as a viral vector, and the vector can be introduced into appropriate cells in an animal. In such a method, the cell population can be engineered to inducibly or constitutively express active galanin or an analog thereof.

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

Direct injection of a nucleic acid molecule alone or encapsulated, for example, in cationic liposomes may be used for stable gene transfer of a polynucleotide molecule comprising a galanin gene into non-dividing or dividing cells in vivo (Ulmer et al., Science 259:1745-1748 (1993)). In addition, the polynucleotide molecule can be transferred into a variety of tissues in vivo using the particle bombardment method (Williams et al., Proc. Natl. Acad. Sci. USA 88:2726-2730 (1991)).

Viral vectors are also useful for transfer of polynucleotide molecule comprising a galanin encoding region into specific cell types in vivo. Viruses are specialized infectious agents that can infect and propagate in specific cell types. The selection of a viral vector will depend, in part, on the cell type to be targeted. Suitable viral vectors may include, for example, recombinant adeno-associated viral vectors having general or tissue-specific promoters (Lebkowski et al. U.S. Pat. No. 5,354,678).

Gene transfer to obtain expression of a galanin gene in an individual can be performed by, for example, by ex vivo transfection of autologous cells. Suitable cells for such ex vivo transfection include blood cells since these cells are readily accessible for manipulation and reintroduction back into the subject by methods well known in the art. Gene transfer through transfection of cells ex vivo can be performed by a variety of methods, including, for example, calcium phosphate precipitation, diethylaminoethyl dextran, electroporation, lipofection, or viral infection. Such methods are well known in the art (see, for example, Sambrook et al., Molecular Cloning: A Laboratory Manual, Cold Springs Harbour Laboratory Press (1989)). Once the cells are transfected, they are then transplanted or grafted back into a subject to be treated.

A method for direct gene transfer into the ruminant mammary gland, which may be useful in the context of the present invention, is described in U.S. Pat. No. 5,780,009. The method involves infusing a liquid complex including a genetic construct into a ductal tree of the mammal. The liquid complex can be infused before the mammal reaches sexual maturity and after the mammal develops a functional streak canal. Alternatively, a liquid complex that is free of live retroviruses can be infused. If desired, the infused genetic construct can be treated with a polycationic compound and/or a lipid to improve the efficiency with which it is taken up by an epithelial cell of the mammary gland.

### Production of Transgenic Animals

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Advances in technologies for embryo micromanipulation now permit introduction of heterologous DNA into, for example, fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals. Those techniques as well known. See reviews of standard laboratory procedures for microinjection of heterologous DNAs into mammalian fertilized ova, including Hogan et al., Manipulating the Mouse Embryo, (Cold Spring Harbor Press 1986); Krimpenfort et al., Bio/Technology 9:844 (1991); Palmiter et al., Cell, 41: 343 (1985); Kraemer et al., Genetic manipulation of the Mammalian Embryo, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated herein by reference

Another method used to produce a transgenic animal involves microinjecting a nucleic acid into pro-nuclear stage eggs by standard methods. Injected eggs are then cultured before transfer into the oviducts of pseudopregnant recipients. Transgenic animals may also be produced by nuclear transfer technology as described in Schnieke, A. E. et al., 1997, Science, 278: 2130 and Cibelli, J. B. et al., 1998, Science, 280: 1256. Using this method, fibroblasts from donor animals are stably transfected with a plasmid incorporating the coding sequences for a binding domain or binding partner of interest under the control of regulatory. Stable transfectants are then fused to enucleated oocytes, cultured and transferred into female recipients.

Analysis of animals which may contain transgenic sequences would typically be performed by either PCR or Southern blot analysis following standard methods.

By way of a specific example for the construction of transgenic mammals, such as cows, nucleotide constructs comprising a sequence encoding a binding domain fused to GFP are microinjected using, for example, the technique described in U.S. Pat. No. 4,873,191, into oocytes which are obtained from ovaries freshly removed from the mammal. The oocytes are aspirated from the follicles and allowed to settle before fertilization with thawed frozen sperm capacitated with heparin and prefractionated by Percoll gradient to isolate the motile fraction.

The fertilized oocytes are centrifuged, for example, for eight minutes at 15,000 g to visualize the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals, such as cattle, by administering coprostanol. Oestrous is produced within two days and the embryos are transferred to the recipients 5-7 days after estrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741.

The present invention is further described by the following non-limiting Examples.

### Experimental Procedures

### Animals

Gal^{-/-} mice (17) used in these studies were of the 129OlaHsd genetic background. Rag1^{-/-} mice (21) on the inbred C57BL/6J background were purchased from Animal Resource Centre, Perth, Australia. All animals were specific pathogen free and housed with food and water ad libitum with a 12 hr day/night cycle at 22° C. and 80% relative humidity.

mRNA Isolation

The 4^{th} inguinal mammary gland was frozen in liquid nitrogen before storage at −80° C. prior to use. Total RNA was extracted using TRIZOL Reagent (Gibco BRL) according to the manufacturer's instructions.

### Reverse Transcription Polymerase Chain Reaction (RT-PCR)

First strand cDNA synthesis used avian myeloblastosis transcriptase (Promega) according to the manufacturer's instructions. PCR primers for Galanin (Acc No. NM 010253), Galr1 (Acc No. NM 008082), Galr2 (Acc No. NM 010254), Galr3 (Acc No. NM 015738) and GAPDH (Acc No. M32599) were designed on the basis of mismatch to other genes. The following primers were used in this study: (Galanin) mGal-F15′-TGCAGTAAGCGACCATCCAG-3′ (forward) (SEQ ID NO:17) and mGal-R15′-AGCACAGGACACACGTGCAC-3′ (reverse) (SEQ ID NO:18), (Galr1) mGalr1-F15′-CGCCTTCATCTGCAAGTTTA-3′ (forward) (SEQ ID NO:19) and mGalr1-R15′-CAGGACGGTCTGTGCAGT-3′ (reverse) (SEQ ID NO:20), (Galr2) mGalr2-F1 5′-TGCCTTTCCAGGCCACCATC-3′ (forward) (SEQ ID NO:21) and mGalr2-R15′-GCGTAAGTGGCACGCGTGAG-3′ (reverse) (SEQ ID NO:22), (Galr3) Galr3-F1 5′-CCTGGCTCTTTGGGGCTTTCGTG-3′ (forward) (SEQ ID NO:23) and Galr3-R15′-AGCGCGTAGAGCGCGGCCACTG-3′ (reverse) (SEQ ID NO:24), (GAPDH) GAPDH-F1 5′-TGACATCAAGAAGGTGGTGAAGC-3′ (forward) (SEQ ID NO:25) and GAPDH-R15′-AAGGTGGAAGAGTGGGAGTTGCTG-3′ (reverse) (SEQ ID NO:26). The amplification regime consisted of a 94° C. 10 min denaturation cycle, followed by 94° C. for 25 sec, 58° C. for 30 sec, and 72° C. for 2 min, for 33 cycles. An elongation step of 72° C. for 5 min ended the PCR. Oligonucleotides for internal hybridisation of PCR products were 5′-AATGGCCACGTAGCGATCCA-3′ (Galr1) (SEQ ID NO:27), 5′-GTAGCTGCAGGCTCAGGTTCC-3′ (Galr2) (SEQ ID NO:28) and 5′-GTGGCCGTGGTGAGCCTGGCCT-3′ (Galr3) (SEQ ID NO:29).

### Recombined Mammary Gland Transplantation

Donor mammary tissue (1 mm³) from Gal^{+/+} or Gal^{-/-} 12 week old mice was inserted into the excised fat pad of Gal^{+/+} or Gal^{-/-} 3 week old mice cleared of endogenous epithelium. This recombined mammary epithelium-stroma complex was then grafted between the abdominal cavity and skin, between the 3^{rd} and 4^{th} mammary glands of 3 week old Rag1^{-/-} mice (22). This procedure resulted in 100% transplant survival with >95% showing ductal outgrowth. Using this method, recombinations of mammary epithelium and stroma were produced that allowed deletion of the galanin gene from stroma and/or epithelium.

### Histological Analysis

Mammary whole mounts were made by spreading the gland on a glass slide and fixing in 10% formalin solution. Glands were defatted in acetone before carmine alum (0.2% carmine, 0.5% aluminium sulfate) staining overnight. The whole mount was dehydrated using a graded ethanol series followed by xylene treatment for 60 min and storage and photography in methyl salicylate. Morphometric analysis was performed by counting the number of side branches, alveolar buds or lobuloalveoli per mammary gland (n=5) for mammary gland cultures or from 4 representative fields of view from whole 4^{th} inguinal glands.

### Prl Treatment of Mice

On the morning of the observation of a vaginal plug, 6-8 week old mice were implanted with a 0.25 μl per hour, 28 day mini-osmotic pump (Alzet) containing unmodified Prl prepared as described (23). Either 0.6 or 1.2 μg were delivered per 24 hr. On the first day post-partum maternal behaviour of mothers was observed, pups were examined for the presence of milk and glands were taken for histological analysis.

### Mammary Gland Culture

Four week old BALB/c mice were implanted with estrogen, progesterone and cholesterol pellets (Ginsburg and Vonderhaar, 2000). Following nine days of treatment, the whole fourth glands were removed and stretched onto siliconized lens paper and placed into petri dishes containing 2 mL of Waymouths 152/1 medium supplemented with penicillin (100 U/ml), streptomycin (100 μg/ml), gentamycin sulfate (50 μg/ml), 20 mM HEPES, insulin (5 μg/ml), hydrocortisone (100 ng/ml) and aldosterone (100 ng/ml) (basal medium, IAH) to monitor ductal side branching, with and without 100 nM rat galanin (Auspep). To assess lobuloalveolar development ovine PRL (Sigma, 1 μg/ml) was added to the basal medium with or without galanin. Glands were maintained in a tri-gas incubator at 50% O₂ and 5% CO₂ in air. Medium was changed after 24 hr, then every second day for 6 days before morphology and histology were assessed.

### Transcript Profiling

Total RNA was extracted using TRIZOL Reagent (Gibco BRL), purified using RNeasy Mini Kit (QIAGEN), cDNA synthesis performed using Superscript II (Invitrogen Life Technologies), synthesis of Biotin-labeled cRNA performed using BioArray HighYield RNA Transcript labeling Kit (Enzo Diagnostics) and hybridised to Affymetrix MGU74v2 GeneChips overnight as per manufacturers instructions. Arrays were performed in duplicate using 4-6 glands per treatment group from two separate replicate experiments. Analysis was performed using the Affymetrix GeneChip v5 software (MAS 5), with treatment groups compared back to IAH treatment as the baseline comparison. Principal Components Analysis was performed using JMP (SAS Institute). Venn diagrams where formed by selecting genes called increasing or decreasing by MAS 5 with a fold change greater then 1.7 compared to IAH. These groups were further restricted by excluding genes with a magnitude fold change >1.2 induced by the other treatments.

### Quantitative RT-PCR

Quantitative PCR was performed using LightCycler technology (Roche). Primers were designed on the basis of mismatch to other genes for WDMN1, β-Actin, WAP, β-casein, 8-casein, Elf5, Glycam1, IGF1, GHR, SPOT 14 and PRLR. PCR reactions were performed in 10 μL volume with 1 μL of cDNA, 5 pmoles of each primer and FastStart DNA Master SYBR Green I enzyme mix (Roche) as per manufacturers instructions. Relative quantitation of the product was performed by comparing the crossing points of different samples normalised to an internal control (β-Actin). Each cycle in the linear phase of the reaction corresponds to a two fold difference in transcript levels between samples. Each reaction was performed in triplicate using pooled RNA from the 4-6 mammary glands or the treatment groups utilised for transcript profiling.

### Western Analysis

Following RNA extraction from mammary glands using TRIZOL Reagent (Gibco BRL), protein was extracted following the manufacturer's instructions. Protein was separated using SDS-PAGE (Bio-Rad Laboratories), transferred to PVDF (Millipore) and blocked overnight with 5% skim milk powder, 2% fetal bovine serum, 50 mM sodium phosphate, 50 mM NaCl and 0.1% Tween 20. Membranes were incubated with one of the following primary antibodies: α-milk protein (Accurate Chemical & Scientific Corporation), α-STAT5a (Upstate Biotech), α-phospho-STAT5, α-phospho-Erk1/2, α-Erk2, α-phospho-Akt (S473), α-phospho-Akt (T308), α-Akt (Cell Signaling Technology) or α-β-Actin (Sigma). 20 μg of protein was loaded per lane except for α-milk protein were 400 ng of protein was loaded. Specific binding was detected using Horseradish peroxidase conjugated secondary antibodies (Amersham Biosciences) with Chemiluminescence Reagent (PerkinElmer) and Biomax Light Film (Eastman Kodak Company).

### EXAMPLE 1

### Prolactin Supplementation of Gal^{-/-} Mice Allows Lactation Sufficient for Pup Survival but Lobuloalveolar Development in not Completely Rescued

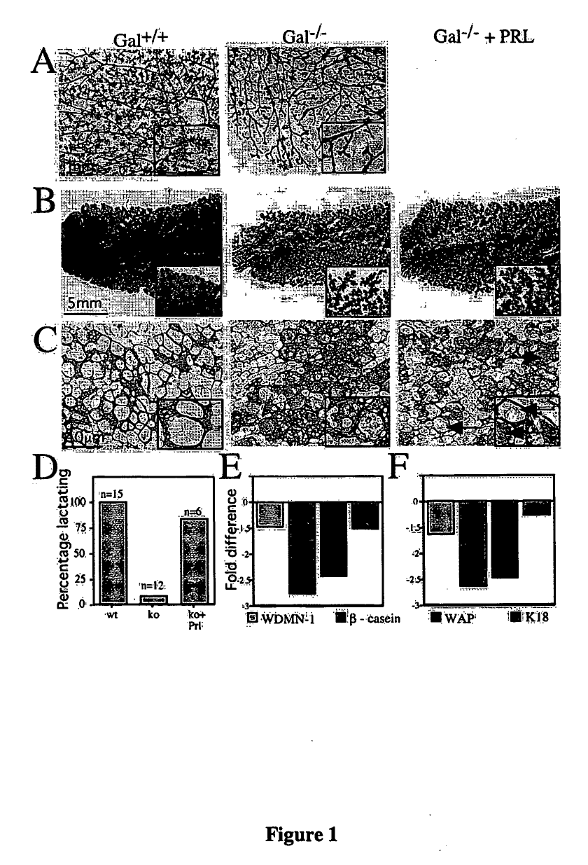

Targeted disruption of the galanin gene results in failure of ductal side branching during puberty and lactational failure following the first pregnancy. We have previously ascribed this effect to the reduced levels of serum prolactin seen in Gal^{-/-} animals, but this hypothesis has not been tested and the defects in the mammary glands of Gal^{+/+} have not been investigated during pregnancy (17). At day 12 of pregnancy the size and density of lobuloalveoli were decreased in Gal^{-/-} mammary glands compared with that in galanin wild type (Gal^{+/+}) mice (FIG. 1A). This defect continued throughout pregnancy and at the 1^{st} day post-partum, Gal^{-/-} mammary glands showed reduced lobuloalveolar density compared to Gal^{+/+} mice (FIG. 1B). Histological examination showed that although the lobuloalveoli had formed, lactation had not commenced in Gal^{-/-} mice (FIG. 1C). Examination of the stomach contents of the pups for milk showed that 11 of 12 knockout females were unable to lactate following their first pregnancy (FIG. 1D), despite the observation of normal maternal behaviour and suckling of pups. Differentiation of the mammary epithelium was assessed by quantitative analysis of the mRNA levels of several milk protein genes. Early (WDMN-1), mid (β-casein) and late (WAP) stage markers of epithelial cell differentiation were all decreased in Gal^{-/-} mammary glands compared to Gal^{+/+} littermates (FIG. 1E). Epithelial content was assessed by quantitative measurement of keratin18 mRNA levels and showed similar levels in Gal^{+/+} and Gal^{-/-} glands (FIG. 1E). This finding, combined with the histological findings, indicate that the lobuloalveoli had formed in Gal^{-/-} mammary glands, but that differentiation and lactogenesis had failed. Thus the reduced area of epithelium apparent in the Gal^{-/-} whole mounts and histology at term is due to a failure of lobuloalveolar engorgement due to failed onset of milk secretion, but not to a detectable decrease in epithelial cell number. A similar defect is seen in PRLR^{+/-} mice (4), and interestingly this effect was lost following their second pregnancy, as seen in Gal^{-/-} mice (24).

Since homozygous disruption of the galanin gene results in decreased levels of plasma PRL during pregnancy, we determined whether treatment of Gal^{-/-} mice with PRL would rescue the defect in lobuloavleolar development and lactation. Using a mini osmotic pump, treatment with either 0.6 or 1.2 μg of PRL per 24 hours throughout the duration of pregnancy restored lactation and allowed pup survival (FIG. 1D). Mammary gland whole mounts showed a partial rescue of lobuloalveolar density (FIG. 1B), but histological examination showed that many alveoli had not commenced lactation, identified by their retention of highly proteinaceous (pink staining) contents (FIG. 1C). Analysis of milk protein gene expression revealed that PRL treatment did not even partially rescue the defect in lobuloalveolar differentiation measured by the expression of the milk proteins WDMN1 and β-casein, and WAP, despite allowing pup survival (FIG. 1F). Keratin18 levels were unchanged by treatment with PRL. Thus, although PRL treatment of Gal^{-/-} mice restored lactation to a level sufficient for pup survival, it failed to produce any detectable rise in milk protein gene expression. If the effects on lactation seen in Gal−/− mice were only mediated via pituitary prolactin secretion then we would have expected to see some rise in milk protein levels given the rescue of lactation. Thus it is unlikely that our failure to rescue milk protein expression is due solely to insufficient administration of prolactin, and we conclude that galanin acts to influence mammary differentiation via a mechanism additional to the regulation of prolactin secretion. We have investigated this hypothesis further.

### EXAMPLE 2

### Galanin and Galanin Receptors are Differentially Expressed in the Mammary Gland

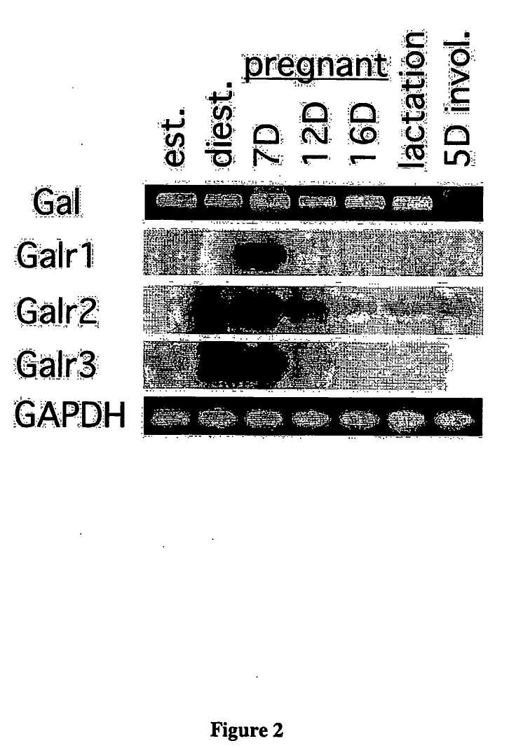

The failure to rescue milk protein expression with PRL treatment of Gal^{-/-} mice indicated that galanin may act by an additional mechanism to regulate mammary epithelial cell differentiation. Expression of mRNA for galanin and Galr 1-3 was examined by RT-PCR using mouse mammary glands collected at various stages of development (FIG. 2). The galanin transcript was expressed at all time points; from estrous in nulliparous mice through to lactation, but was not detected during involution. Expression of transcripts for the galanin receptors was tightly regulated and coordinated. Transcripts for all three receptors were most highly expressed at day 7 of pregnancy. Galr1 transcripts were only detected at this time, while Galr2 mRNA was also detected at lower levels throughout the later stages of pregnancy and involution, and Galr3 mRNA was also detected during estrous and diestrous in the nulliparous mice. Very low expression of Galr3 mRNA could also be detected at 5 days of involution with longer exposure (data not shown).

The coordinated regulation of galanin receptors in the mammary gland and increase of galanin in serum during pregnancy suggested a possible endocrine role for galanin, while the expression of galanin in the mammary gland also raised the possibility of an autocrine or paracrine mechanism.

### EXAMPLE 3

### An Autocrine or Paracrine Mechanism of Galanin Action is not Essential for Mammary Gland Development

To determine if galanin produced by the mammary gland was necessary for normal development, we employed mammary epithelial transplantation. Donor mammary epithelium from mature Gal^{+/+} or Gal^{-/-} mice was inserted into the excised fat pad of 3 week old Gal^{+/+} or Gal^{-/-} mice cleared of endogenous epithelium. The recombined mammary epithelium-stroma complex was grafted between the skin and abdominal cavity of 3 week old Rag1^{-/-} mice (22). This allowed deletion of the galanin gene from the stroma and/or epithelium in the context of a normal endocrine background including normal circulating prolactin and galanin levels.

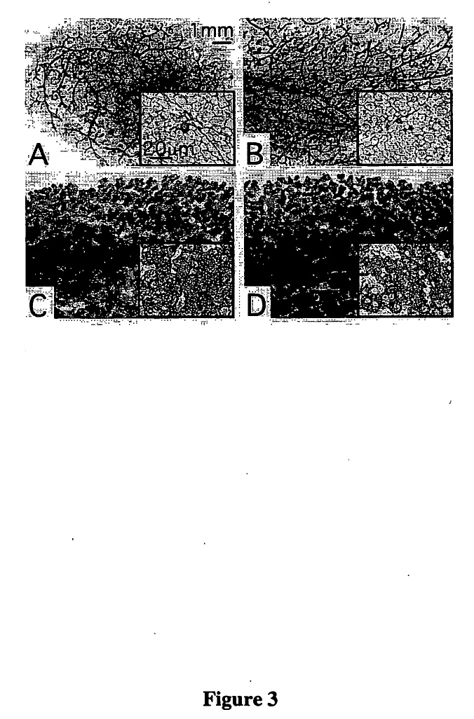

Ablation of galanin from the stroma, epithelium, or from both, did not recapitulate the failure of ductal side branching observed in nulliparous Gal^{-/-} mice, nor the impaired lobuloalveolar development seen on the 1^{st} day post-partum (FIG. 3 and data not shown). These data demonstrate that an autocrine/paracrine role for galanin is not essential for mammary development in the context of normal levels of circulating galanin. Thus endocrine galanin is sufficient for normal mammary gland development in the absence of mammary produced galanin. Mammary produced galanin could, however, play a role in pathological situations where endocrine galanin levels become deficient.

### EXAMPLE 4

### Galanin Can Act Directly on the Mammary Gland to Induce Lobuloalveolar Development

Galanin may act in an endocrine manner via mammary galanin receptors to induce lobuloalveolar development. As galanin treatment in vivo would indirectly induce mammary development via endocrine regulation of PRL and progesterone, we utilised an in vitro mammary gland culture model of mammogenesis (25).

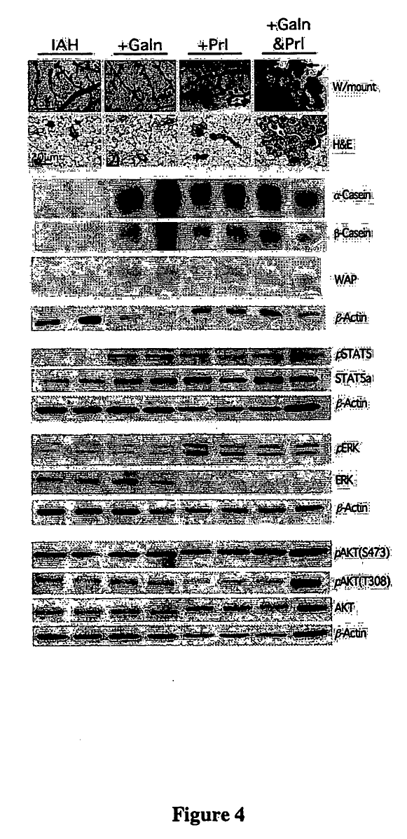

Ductal side branching similar to that seen during puberty was produced when mammary glands were cultured in insulin (I), aldosterone (A) and hydrocortisone (H) (FIG. 4). The addition of 100 nM galanin to the IAH containing medium did not alter ductal or lobuloalveolar development measured by quantitative morphology and histology. When PRL was added to the culture medium, lobuloalveolar development was observed (FIG. 4), although as noted previously, not to the extent observed during pregnancy. The addition of 100 NM galanin to IAH+PRL medium resulted in a 3.8 fold increase in the number of lobuloalveoli per gland (8.6±2.1 IAH+PRL v. 33.0±6.1 IAH+PRL+galanin, p=0.005), causing the glands to resemble those observed during pregnancy. Additionally, the size of individual lobuloalveoli in IAH+PRL+galanin treated glands was also greater than in LAH+PRL treated glands (FIG. 4). These data show that galanin can act directly on the mammary gland to augment PRL-mediated lobuloalveolar development, establishing galanin as a new endocrine factor active during this phase of development.

### EXAMPLE 5

### Galanin Action on Mammary Gland Differentiation Results in Activation of STAT5

To investigate the mechanisms behind the induction of lobuloalveolar development by galanin we examined activation of the JAK/STAT, MAP kinase and PI3 kinase signaling pathways by PRL and galanin. As expected, in mammary glands treated with PRL we saw an increase in total STAT5a and a dramatic increase in phosphorylated STAT5 in these glands (FIG. 4). Similarly, PRL treatment resulted in the sustained activation the MAP kinase pathway. While the level of total ERK1/2 decreased, the levels of phosphorylated ERK dramatically increased in mammary glands exposed to PRL. Examination of PI3 kinase signalling revealed decreased mobility but no increase in total Akt in glands receiving PRL. PRL did not increase phosphorylation of T308 and S473 residues of Akt, those most commonly associated with Akt activation. The decrease in mobility may represent phosphorylation of other sites on the Akt molecule.

Surprisingly, galanin treatment alone resulted in activation of the JAK/STAT pathway, similar to PRL (FIG. 4), but in stark contrast to PRL, galanin did not induce sustained activation of the MAP kinase pathway or alter the mobility of Akt. When mammary glands were treated with galanin and PRL there were no dramatic changes to the effects produced by either hormone alone. The apparent slight diminution in pERK and increase in Akt and pAkt (T308) in the figure were not consistent between experimental replicates.

We examined markers of mammary epithelial cell differentiation by western blot. Again as expected, mammary glands treated with PRL synthesised the milk proteins WAP, α and β casein. Strikingly, galanin alone produced induction of milk protein synthesis despite failing to induce lobuloalveolar development (FIG. 4).

These results show that galanin can induce epithelial cell differentiation, as measured by milk protein synthesis, and the sustained activation of the JAK/STAT pathway. Whether galanin acts directly via its receptors to activate Stat5, or whether this effect is indirect remains a question for further investigation. The salient point is that galanin treatment caused sustained activation of the Stat5 pathway and cell differentiation measured by milk protein expression. In contrast, prolactin caused sustained activation the JAK/STAT, and the additional activation of the MAP kinase and possibly PI3 kinase pathways (and is known to activate these pathways directly), and induced both epithelial cell differentiation and epithelial cell proliferation. Together these hormones have a synergistic effect and allow lobuloalveolar development to proceed in vitro to a level beyond that achievable by PRL alone.

In conclusion, Gal^{-/-} mammary glands show failed differentiation despite prolactin supplementation and wild type glands in organ culture show differentiation in response to galanin alone. These observations establish galanin as a hormone with potent ability to produce differentiation of the mammary epithelium.

### EXAMPLE 6

### Transcriptional Profiling of Galanin and Prolactin Induced Mammary Development

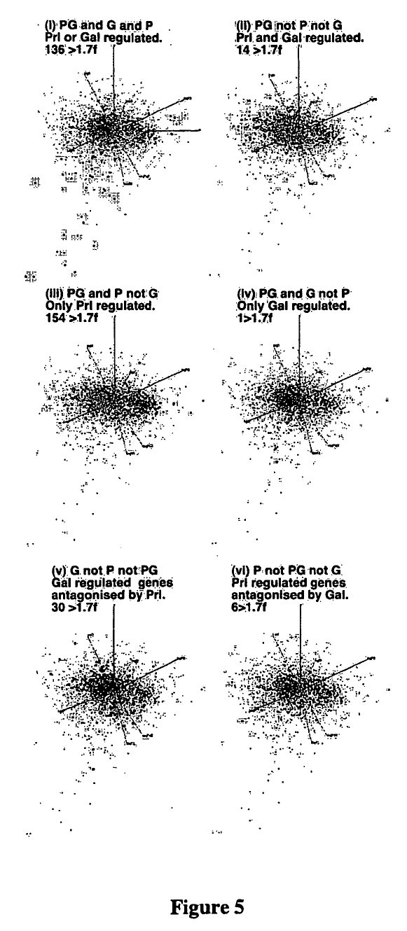

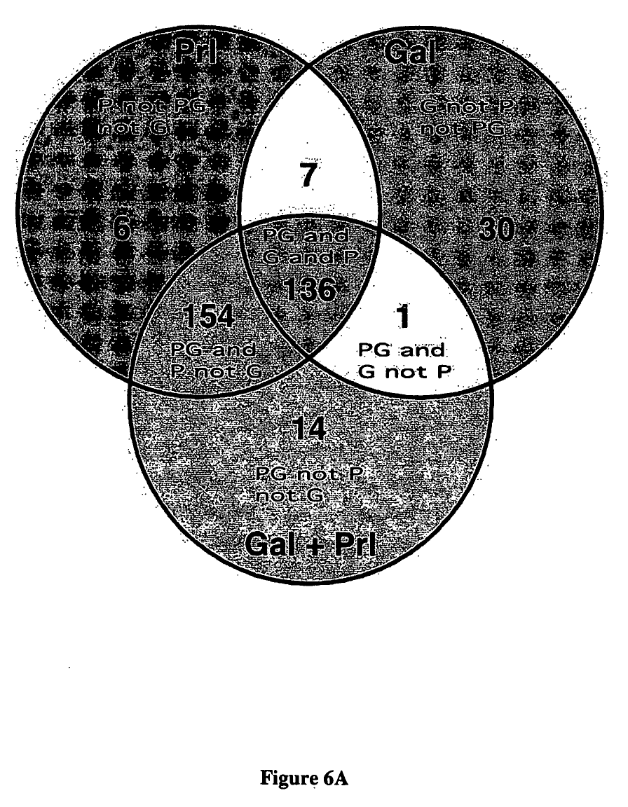

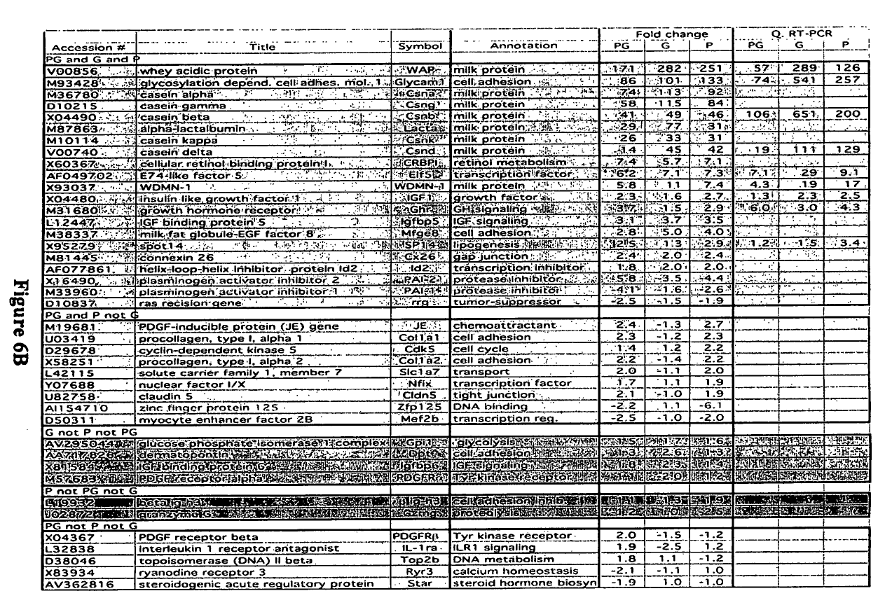

To examine the nature of the transcriptional interaction between prolactin and galanin which controls mammary gene expression during lactogenesis, we measured the transcriptional response of the cultured mammary glands shown in FIG. 4 to galanin, PRL and PRL+galanin using the Affymetrix microarray suite and MGU74Av2 oligonucleotide GeneChips. A principal components analysis of this data is shown in FIG. 5, using a Venn-Diagram approach to define sets of genes that were regulated by the three treatments. The numbers of genes and identities of the genes within these sets are shown in FIG. 6.

We used principal components analysis to distinguish patterns of altered gene expression (FIG. 5). Here, genes with a decreased expression relative to LAH are coloured red and those with increased expression are coloured green. No arbitrary fold change value was applied in this analysis. It is apparent from this analysis that a strikingly complex and non-symmetrical transcriptional interaction exists between galanin and prolactin during lobuloalveolar differentiation. Three major sets of genes showed a robust response.

The first major set of genes we identified comprises genes that changed expression in response to all three treatments; PRL, galanin PRL+galanin (FIG. 5i). The genes are thus regulated independently by galanin or PRL. Use of an arbitrary fold change value of 1.7 reveals 136 genes (40% of all regulated genes) in this set (FIG. 6A). Genes with increased expression in this set (FIG. 6B) include markers of mammary epithelial cell differentiation, such as the milk proteins (WAP, WDMN-1 and 5 casein family members). Others here include CIS and SOCS2, negative regulators of the JAK/STAT signalling pathway, providing functional demonstration of activation of the JAK/STAT pathway by galanin and prolactin. Genes with demonstrated roles in mammary development are also independently regulated by both galanin or PRL. These include E74-like factor 5 (Elf5), growth hormone receptor (GHR), insulin-like growth factor 1 (IGF-1), IGF binding protein 5 (IGFBP-5) and helix-loop-helix protein Id2 (2, 26-29). Galanin did not induce PRL or PRLR gene expression and prolactin did not regulate galanin or its receptors, excluding this simple mechanism for these transcriptional effects.

A second interesting subset of genes (FIG. 5ii) with 14 members with fold a change >1.7 were regulated by treatment using PRL+galanin but not by treatment with either hormone alone, identifying a synergistic effect of these two hormones that is consistent with the synergistic effect of galanin and PRL on lobuloalveolar development. Almost all genes in this set showed an increase in expression indicating an overwhelmingly positive transcriptional effect of these hormones in synergy. Genes in this group include platelet-derived growth factor receptor beta (PDGFRβ), interleukin 1 receptor antagonist and steroidogenic acute regulatory protein (FIG. 6B).

A third major set (FIG. 5iii) contains 154 regulated genes which change greater than 1.7 fold and is found at the intersection of the PRL and PRL+galanin treatment groups. This group of genes are regulated by PRL regardless of the presence of galanin. Genes in this group include procollagen 1 alpha 1 & 2, nuclear factor I/X, claudin 5, and zinc finger protein 125. In striking contrast, the reciprocal set of genes at the intersection of the galanin and PRL+galanin treatment groups (FIG. 5iv) contains just one gene with a change of 1.7 fold or more, although thirty genes are regulated by galanin when PRL is not present (FIG. 5v). This asymmetry indicates firstly that prolactin has a much greater unique transcriptional influence that galanin, regulating 160 genes that are not regulated by galanin, compared to 31 genes that are regulated by galanin but not prolactin. Secondly prolactin acts to antagonise almost all (30/31 genes) of the unique influence of galanin. Galanin does not have the same effect on prolactin induced gene expression, as only 6 of 160 genes show prolactin regulated expression that was antagonised by galanin (FIG. 5vi). Genes found in these asymmetric sets include IGF-binding protein 6 (IGFBP-6), platelet-derived growth factor receptor alpha (PDGFRα), dermatopontin and glucose phosphate isomerase 1 (FIG. 6B).

These transcript profiles show that galanin and prolactin interact in the mammary gland to control gene expression via mechanisms that are independent, common, antagonistic and synergistic.

### EXAMPLE 7

### Mouse Models for Determining Effects of Increased Galanin or Galanin Analogues on Susceptibility to Mammary Cancer

A transgenic mouse which over-expresses galanin can be used to determine preferred conditions for methods of treating mammary hyperproliferative diseases, such as breast cancer, that are based on administration of galanin or analogs thereof. The mouse mammary tumour virus (MMTV) long terminal repeat is currently the preferred promoter to ensure mammary specific expression that does not require pregnancy for expression. This promoter may be used to drive the expression of the galanin gene contained in a mouse genomic DNA fragment, which will ensure high expression of galanin. An internal ribosome entry site in the construct will also allow the production of the EGFP protein, allowing detection of cells expressing galanin by fluorescent microscopy.

The transgenic mouse will show constitutive expression of galanin in the mammary glands and will be used to examine the consequences of raised galanin levels on mammary gland development, lactational performance and susceptibility to mammary cancer in response to normal environmental factors and the introduction of carcinogenic insult via chemical (eg DMBA), radiological (eg ionizing radiation) or genetic means (introduction of oncogenic transgenes).

A second construct, which is identical to the MMTV-Galn construct but with MMTV replaced by the tetO sequence, can also be used to generate a transgenic mouse. This construct will allow control of galanin expression by the administration of Doxycycline to animals carrying this construct and an additional MMTV-rtTA construct.

This model will allow the mammary expression of galanin to be raised and lowered at any time. It can be used for similar studies to those outlined above, but will allow better control of the time of galanin expression. For example the consequences of increasing galanin expression in established tumors, or at defined stages of lactation can be investigated. Similarly, the effects of a reduction in galanin levels can be examined during lactation or in tumors established in the presence of high galanin expression.

### Discussion

Experiments detailed in this study show that mammary epithelial cell differentiation was impaired in galanin knockout mice and could not be rescued by prolactin supplementation that was sufficient to allow pup survival. Experiments with mammary glands in culture demonstrated that galanin alone could act directly on the mammary gland to greatly enhance cell differentiation. The combination of galanin and prolactin produced lobuloalveolar development in vitro resembling that seen in whole animals, the first demonstration in vitro of development to this extent. Galanin caused sustained activation of the STAT5 pathway while prolactin caused sustained activation of the STAT5, MAP kinase and possibly Akt pathways. Analysis of the transcriptional results of this differential activation of signaling pathways showed the interaction between prolactin and galanin had independent, common, antagonistic and synergistic components.

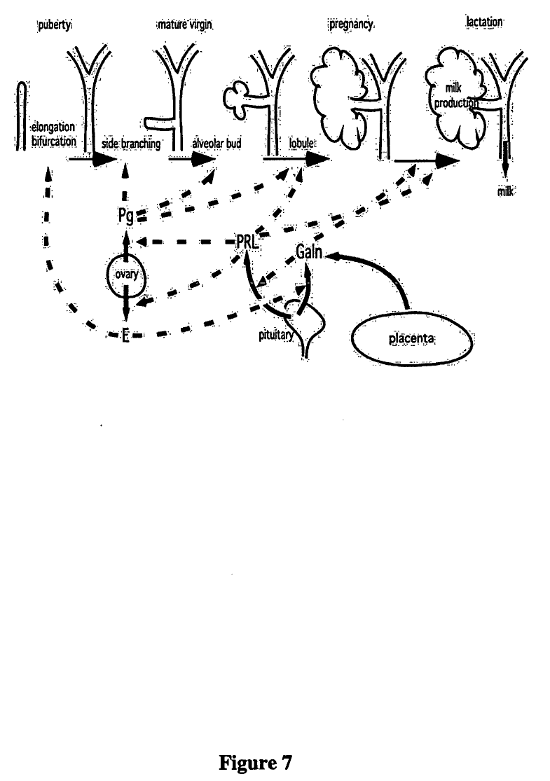

A major finding from these experiments is that galanin exerts an endocrine effect on mammary gland development. A model of the proposed endocrine role for galanin in mammary gland development is presented in FIG. 7, where galanin is proposed to exert both indirect and direct effects. The indirect effects stem from galanin's action as a growth factor to the lactotroph—the PRL producing cells of the pituitary. Via this mechanism galanin controls the level of circulating PRL (17), which in turn acts both indirectly and directly to control ductal side branching and lobuloalveolar development respectively (4,5,30). Galanin's direct effects on the mammary gland arise from its presence in the circulation, derived from both the pituitary and the placenta (20). Placental production of hormones represents a mechanism by which the developing foetus can “hijack” the maternal endocrine system to ensure its nourishment and survival (31). Our results suggest that this may be extended to the preparation of the mammary gland for lactation via the influence of placental galanin.

A second major finding from these experiments is that galanin can produce sustained activatation of the Stat5 signaling pathway. This pathway is essential for lobuloalveolar development and differentiation (32) and is directly activated by receptors for prolactin, growth hormone and epidermal growth factor (2). Stat5 also shows decreased DNA binding activity in the developmentally deficient mammary glands of Id2^{-/-} mice (28). It is clear that activation of STAT5 represents the convergence point of many different pathways critical for lobuloavleolar development. Galanin, via mechanisms that remain to be fully defined, is capable of producing sustained activation of this pathway.

PRL but not galanin caused sustained activation of the MAP kinase signalling pathway and reduced total MAP kinase levels. MAP kinase has a role in the regulation of cell proliferation and coordinates the mitogenic response of many growth factor—receptor tyrosine kinase induced signalling events, many of which have a role as regulators of proliferation in the mammary gland (33).

The sustained activation observed in these experiments may reflect the changes in cellular differentiation state elicited by these hormones in addition to direct effects, and so we cannot exclude the possibility that galanin causes transient MAP kinase activation, or an indirect action of galanin on Stat5 phosphorylation, but regardless of this caveat these studies demonstrate that galanin alone, like prolactin alone, can cause sustained activation of Stat5 but cannot produce the sustained activation of MAP kinase produced by prolactin. Galanin therefore does not produce an ongoing and major proliferative stimulus, but does produce a strong differentiation signal. This is consistent with galanin's effects on milk protein expression and with the failure of differentiation seen in Gal^{-/-} animals. This indicates that galanin acts as a tumour suppressor gene in the mammary gland.

GeneChip microarrays were used to examine changes in gene expression in the mammary gland following exposure to galanin, PRL, and galanin plus PRL. A striking pattern of gene regulation was observed. The majority of regulated genes fell into three major groups.

The first group showed regulation of expression by all three treatments (PRL, galanin, PRL+galanin), indicating that galanin and PRL both act to control the expression of genes in this group without interaction. From our analysis of signal transduction pathways we would expect this set to contain genes predominantly regulated via the STAT5 pathway, and this set contained the milk protein genes, markers of mammary epithelial differentiation and known JAK/STAT target genes. This group also includes members of the GH/IGF axis-GHR, IGF-1 and IGFBP-5. GHR and IGF-1 have well documented roles in the regulation of ductal growth and milk protein expression (2,38). In whole animals galanin may regulate pituitary GH synthesis and release (39,40) with potential for the regulation of both systemic and local IGF-1 production. IGFBP-5 is a negative regulator of IGF-1 and controls apoptosis in the mammary gland (27).

The second group of genes showed regulation only in response to galanin and prolactin, demonstrating the synergistic regulatory action of prolactin and galanin. Presumably galanin action via its G-protein coupled receptors, combined with the prolactin receptor stimulated pathways are responsible for this synergy. Of particular interest is the synergistic induction of PDGFRβ. While the role of PDGF in normal mammary gland development is unclear, PDGF is a potent mitogen for a variety of different cells including some mammary cells suggestive of a proliferative role for PDGFRP in the mammary gland (41).

The third main group of genes (154 genes) showed regulation of expression by PRL independently of galanin. From our analysis of the signalling pathways activated we would expect this group to be transcriptional targets of sustained activation of the MAP kinase and/or PKB signalling pathways. Genes in this group include cell adhesion molecules (procollagen 1 alpha 1 & 2), transcription factors (nuclear factor IX), tight junction proteins (claudin 5), and DNA binding molecules (zinc finger protein 125).

In contrast to the large number of genes regulated by PRL regardless of whether galanin was present (154 genes) only 1 gene was regulated by galanin regardless of the presence of prolactin, and thirty genes were regulated solely by galanin. We conclude that PRL antagonises the regulation of a significant number of galanin regulated genes. For example galanin reduces the expression of PDGFRA, while the addition of PRL prevents galanin from reducing PDGFRA expression, particularly interesting given the synergistic induction of PDGFRP. From these studies we can conclude that the transcriptional basis for the interaction of galanin and prolactin has independent, common, antagonistic and synergistic components.

In summary we have shown that circulating galanin can influence mammary epithelial differentiation. Galanin is therefore a new member of the small list of systemic hormones that control mammary gland development.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

1. A method of inducing differentiation of mammary epithelial cells, the method comprising administering an effective amount of galanin or a functional analog or agonist thereof to the mammary epithelial cells.

2. A method of inducing differentiation of mammary epithelial cells and/or increasing milk production in a mammal, the method comprising increasing the level of galanin or a functional analog or agonist thereof in the mammary tissue of the mammal.

3. (canceled)

4. A method as claimed in claim 2, wherein the level of galanin is increased by administering to a mammal an amount of galanin or a functional analog or agonist thereof effective to induce differentiation of mammary epithelial cells and/or increase milk production in the mammal.

5. A method as claimed in claim 1, wherein the galanin or functional analog or agonist thereof is selected from the group consisting of:
a polypeptide comprising the fragment GWTLNSAGYLLGP (SEQ ID NO:1);
a human galanin polypeptide having the amino acid sequence GWTLNSAGYLLGPHAVGNHRSFSDKNGLTS (SEQ ID NO:2) or a functional equivalent thereof or a functional fragment thereof;
a bovine galanin polypeptide having the amino acid sequence GWTLNSAGYLLGPHALDSHRSFQDKHGLA (SEQ ID NO:3) or a functional equivalent thereof or a functional fragment thereof;
a porcine galanin polypeptide having the amino acid sequence GWTLNSAGYLLGPHAIDNHRSFHDKYGLA (SEQ ID NO:4) or a functional equivalent thereof or a functional fragment thereof;
a rat galanin polypeptide having the amino acid sequence GWTLNSAGYLLGPHAIDNHRSFSDKHGLT (SEQ ID NO:5) or a functional equivalent thereof or a functional fragment thereof;
a galanin analog having the amino the amino acid sequence GWTLNSAGYLLGPHAVNHRSFSDKNGLTS (SEQ ID NO:6) or a functional equivalent thereof or a functional fragment thereof;
a human GALP (1-60) polypeptide having the amino acid sequence APAHRGRGGWTLNSAGYLLGPVLHLPQMGDQDGKRETALEILDLWKAIDGL PYSHPPQPS (SEQ ID NO:11) or a functional equivalent thereof or a functional fragment thereof;
a porcine GALP (1-60) polypeptide having the amino acid sequence APVHRGRGGWTLNSAGYLLGPVLRHPPSRAEGGGKGKTALGILDLWKAIDG LPYPQSQLAS (SEQ ID NO:12) or a functional equivalent thereof or a functional fragment thereof; and
a rat GALP (1-60) polypeptide having the amino acid sequence APAHRGRGGWTLNSAGYLLGPVLHLSSKANGGRKTDSALEILDLWKAIDGL RYSRSPRMT (SEQ ID NO:13) or a functional equivalent thereof or a functional fragment thereof.

6-13. (canceled)

14. A method as claimed in claim 1, wherein the galanin analog is selected from the group consisting of:
(i) Galanin-(2-29) (i.e. deletion of first amino acid);
(ii) Galanin-(3-29) (i.e. deletion of first 2 amino acids);
(iii) Galanin-(1-15) (i.e. deletion of amino acids 16-29/30);
(iv) Galanin-(1-16) (i.e. deletion of amino acids 17-29/30);
(v) M40: galanin-(1-13)-Pro-Pro-Ala-Leu-Ala-Leu-Ala-amide;
(vi) M15 (galantide): Gly-Trp-Thr-Leu-Asn-Ser-Ala-Gly-Tyr-Leu-Leu-Gly-Pro-Gln-Gin-Phe-Phe-Gly-Leu-Met-NH₂ (SEQ ID NO: 13);
(vii) M35: galanin (1-13)-bradykinin (2-9) amide;
(viii) M32: galanin(1-13)-neuropeptide Y(25-36) amide; and
(ix) C7: galanin(1-13)-spantide amide.

15. A method as claimed in claim 1, wherein the galanin analog is an agonist of the GalR2 receptor.

16. A method as claimed in claim 15, wherein the agonist of the GalR2 receptor is a GALP(1-60) polypeptide or galanin(2-16).

17. A method as claimed in claim 2, wherein the level of galanin in the mammary tissue is increased by administering to the mammal an amount of estrogen or a functional analog thereof effective to increase expression of galanin in the mammal.

18. A method as claimed in claim 2, wherein the increase in the level of galanin or a functional analog or agonist thereof is brought about in conjunction with an increase in level or activity of prolactin or an analog thereof.

19. A method as claimed in any claim 18, wherein galanin or an analog or agonist thereof is administered to the mammal in conjunction with prolactin or an analog thereof.

20. A method as claimed in claim 2, wherein the mammal is selected from the group consisting of primates including human beings, cows, sheep, goats, horses, dogs, cats, rabbits, guinea pigs, rats, mice or other bovine, ovine, equine, canine, feline, rodent or murine species.

21. A method of enhancing mammary development in a mammal, the method comprising increasing the level of galanin or a functional analog or agonist thereof and increasing the level of prolactin or an analog thereof.

22. A method as claimed in claim 21 which comprises administering galanin or an analog or agonist thereof in conjunction with prolactin or an analog thereof.

23. A method as claimed in claim 21, wherein the galanin or functional analog or agonist thereof is selected from the group consisting of:
a polypeptide comprising the fragment GWTLNSAGYLLGP (SEQ ID NO: 1);
a human galanin polypeptide having the amino acid sequence GWTLNSAGYLLGPHAVGNHRSFSDKNGLTS (SEQ ID NO:2) or a functional equivalent thereof or a functional fragment thereof;
a bovine galanin polypeptide having the amino acid sequence GWTLNSAGYLLGPHALDSHRSFODKHGLA (SEQ ID NO:3) or a functional equivalent thereof or a functional fragment thereof;
a porcine galanin polypeptide having the amino acid sequence GWTLNSAGYLLGPHAIDNHRSFHDKYGLA (SEQ ID NO:4) or a functional equivalent thereof or a functional fragment thereof;
a rat galanin polypeptide having the amino acid sequence GWTLNSAGYLLGPHAIDNHRSFSDKHGLT (SEQ ID NO:5) or a functional equivalent thereof or a functional fragment thereof;
a galanin analog having the amino the amino acid sequence GWTLNSAGYLLGPHAVNHRSFSDKNGLTS (SEQ ID NO:6) or a functional equivalent thereof or a functional fragment thereof;
a human GALP (1-60) polypeptide having the amino acid sequence APAHRGRGGWTLNSAGYLLGPVLHLPQMGDQDGKRETALEILDLWKAIDGL PYSHPPQPS (SEQ ID NO:11) or a functional equivalent thereof or a functional fragment thereof;
a porcine GALP (1-60) polypeptide having the amino acid sequence APVHRGRGGWTLNSAGYLLGPVLRHPPSRAEGGGKGKTALGILDLWKAIDG LPYPQSQLAS (SEQ ID NO:12) or a functional equivalent thereof or a functional fragment thereof; and
a rat GALP (1-60) polypeptide having the amino acid sequence APAHRGRGGWTLNSAGYLLGPVLHLSSKANGGRKTDSALEILDLWKAIDGL RYSRSPRMT (SEQ ID NO:13) or a functional equivalent thereof or a functional fragment thereof.

24-31. (canceled)

32. A method as claimed in claim 21, wherein the galanin analog is selected from the group consisting of:
(i) Galanin-(2-29) (i.e. deletion of first amino acid);
(ii) Galanin-(3-29) (i.e. deletion of first 2 amino acids);
(iii) Galanin-(1-15) (i.e. deletion of amino acids 16-29/30);
(iv) Galanin-(1-16) (ie. deletion of amino acids 17-29/30);
(v) M40: galanin-(1-13)-Pro-Pro-Ala-Leu-Ala-Leu-Ala-amide;
(vi) M15 (galantide): Gly-Trp-Thr-Leo-Asn-Ser-Ala-Gly-Tyr-Leu-Leu-Gly-Pro-Gln-Gln-Phe-Phe-Gly-Leu-Met-NH₂ (SEQ ID NO: 13);
(vii) M35: galanin (1-13)-bradykinin(2-9) amide;
(viii) M32: galanin(1-13)-neuropeptide Y(25-36) amide; and
(ix) C7: galanin(1-13)-spantide amide.

33. A method as claimed in claim 21, wherein the galanin analog is an agonist of the GalR2 receptor.

34. A method as claimed in claim 21, wherein the agonist of the GalR2 receptor is a GALP(1-60) polypeptide or galanin(2-16).

35. (canceled)

36. A transgenic mammal having integrated in its genome a nucleic acid construct comprising a sequence encoding galanin or an analog thereof, wherein the transgenic mammal expresses galanin or an analog thereof at an elevated level compared to an equivalent non-transgenic mammal, and wherein the level of milk production is increased in the transgenic mammal when compared to an equivalent non-transgenic mammal.

37. A transgenic mammal as claimed in claim 36, wherein the transgenic mammal is a cow, sheep, pig or goat.

38. A transgenic mammal as claimed in claim 36, wherein the sequence encoding galanin is selected from a cDNA sequence as shown in SEQ ID NO:14 (which encodes human galanin) or a fragment thereof, SEQ ID NO:15 or a fragment thereof (which encodes bovine galanin) and SEQ ID NO:16 or a fragment thereof (which encodes porcine galanin).

39. A transgenic mammal as claimed in claim 36, wherein the nucleic acid construct further comprises a mammary specific promoter operably linked to the sequence encoding galanin or an analog thereof.

40. A transgenic mammal as claimed in claim 39, wherein the mammary specific promoter is selected from the group consisting of the WAP promoter, the murine mammary tumour virus (MMTV) long terminal repeat, the new-related lipocalin (NRL) promoter, the betacasein promoter, the beta-lactoglobulin (BLG) promoter and the beta 1,4 galactosyltransferase promoter.

41. A method for the treatment of a mammary hyperproliferative disease and/or inhibiting the growth of a mammary epithelial tumour in a subject, the method comprising administering to the subject an inhibitorially effective therapeutic amount of galanin or a functional analog or agonist thereof.

42. (canceled)

43. A method as claimed in claim 41, wherein the mammary hyperproliferative disease is breast cancer.

44. A method as claimed in claim 41, wherein the galanin or functional analog or agonist thereof is selected from the group consisting of:
a polypeptide comprising the fragment GWTLNSAGYLLGP (SEQ ID NO:1);
a human galanin polypeptide having the amino acid sequence GWTLNSAGYLLGPHAVGNHRSFSDKNGLTS (SEQ ID NO:2) or a functional equivalent thereof or a functional fragment thereof;
a bovine galanin polypeptide having the amino acid sequence GWTLNSAGYLLGPHALDSHRSFODKHGLA (SEQ ID NO:3) or a functional equivalent thereof or a functional fragment thereof;
a porcine galanin polypeptide having the amino acid sequence GWTLNSAGYLLGPHAIDNHRSFHDKYGLA (SEQ ID NO:4) or a functional equivalent thereof or a functional fragment thereof;
a rat galanin polypeptide having the amino acid sequence GWTLNSAGYLLGPHAIDNHRSFSDKHGLT (SEQ ID NO:5) or a functional equivalent thereof or a functional fragment thereof;
a galanin analog having the amino the amino acid sequence GWTLNSAGYLLGPHAVNHRSFSDKNGLTS (SEQ ID NO:6) or a functional equivalent thereof or a functional fragment thereof;
a human GALP (1-60) polypeptide having the amino acid sequence APAHRGRGGWTLNSAGYLLGPVLHLPQMGDQDGKRETALEILDLWKAIDGL PYSHPPQPS (SEQ ID NO:11) or a functional equivalent thereof or a functional fragment thereof;
a porcine GALP (1-60) polypeptide having the amino acid sequence APVHRGRGGWTLNSAGYLLGPVLRHPPSRAEGGGKGKTALGILDLWKAIDG LPYPQSQLAS (SEQ ID NO:12) or a functional equivalent thereof or a functional fragment thereof; and
a rat GALP (1-60) polypeptide having the amino acid sequence APAHRGRGGWTLNSAGYLLGPVLHLSSKANGGRKTDSALEILDLWKAIDGL RYSRSPRMT (SEQ ID NO:13) or a functional equivalent thereof or a functional fragment thereof.

45-52. (canceled)

53. A method as claimed in claim 41, wherein the galanin analog is selected from the group consisting of:
(i) Galanin-(2-29) (i.e. deletion of first amino acid);
(ii) Galanin-(3-29) (i.e., deletion of first 2 amino acids);
(iii) Galanin-(1-15) (i.e. deletion of amino acids 16-29/30);
(iv) Galanin-(1-16) (i.e. deletion of amino acids 17-29/30);
(v) M40: galanin-(1-13)-Pro-Pro-Ala-Leu-Ala-Leu-Ala-amide;
(vi) M15 (galantide): Gly-Trp-Thr-Leu-Asn-Ser-Ala-Gly-Tyr-Leu-Leu-Gly-Pro-Gln-Gln-Phe-Phe-Gly-Leu-Met-NH₂ (SEQ ID NO: 13);
(vii) M35; galanin(1-13)-bradykinin(2-9) amide;
(viii) M32: galanin (1-13)-neuropeptide Y(25-36) amide; and
(ix) C7: galanin(1-13)-spantide amide.

54. A method as claimed in claim 41, wherein the galanin analog is an agonist of the GalR2 receptor.

55. A method as claimed in 41, wherein the agonist of the GalR2 receptor is a GALP(1-60) polypeptide or galanin(2-16).

56. (canceled)
